(19) United States
(12) Patent Application Publication
(10) Pub. No.: US 2016/0272992 A1
(43) Pub. Date: Sep. 22, 2016

(54) PROTECTION OF PLANTS AGAINST OXIDATIVE STRESS

(71) Applicants: VIB VZW, Gent (BE); UNIVERSITEIT GENT, Gent (BE)

(72) Inventors: Lieven De Veylder, Drongen (BE); Toon Cools, Gent (BE)

(21) Appl. No.: 15/037,308

(22) PCT Filed: Nov. 17, 2014
(86) PCT No.: PCT/EP2014/074758

§ 371 Date: May 17, 2016

(30) Foreign Application Priority Data
Nov. 19, 2013 (EP) 13193423.4

(51) Int. Cl. C12N 15/82

(52) U.S. Cl. CPC C12N 15/8271; C12N 15/8218

(57) ABSTRACT

Described is the use of SMR5, possibly in combination with SMR4 and/or SMR7, to modulate ROS and oxidative stress response in plants. More specifically, it relates to an SMR5 knock out or knock down to improve the oxidative stress tolerance in plants.

### CROSS-REFERENCE TO RELATED APPLICATIONS

This application is a national phase entry under 35 U.S.C. §371 of International Patent Application PCT/EP2014/074758, filed Nov. 17, 2014, designating the United States of America and published in English as International Patent Publication WO 2015/074992 A1 on May 28, 2015, which claims the benefit under Article 8 of the Patent Cooperation Treaty to European Patent Application Serial No. 13193423.4, filed Nov. 19, 2013.

### TECHNICAL FIELD

This application relates generally to plant biology and more specifically to the use of SMR5, possibly in combination with SMR4 and/or SMR7, to modulate ROS and oxidative stress response in plants. More specifically, it relates to an SMR5 knock out or knock down to improve the oxidative stress tolerance in plants.

### BACKGROUND

Being immobile, plants are continuously exposed to changing environmental conditions that can impose biotic and abiotic stresses. One of the consequences observed in plants subjected to altered growth conditions is the disruption of the reactive oxygen species (ROS) homeostasis (Mittler et al., 2004). Under steady-state conditions, ROS are efficiently scavenged by different non-enzymatic and enzymatic antioxidant systems, involving the activity of catalases, peroxidases, and glutathione reductases. However, when stress prevails, the ROS production rate can exceed the scavenging mechanisms, resulting into a cell- or tissue-specific rise in ROS. These oxygen derivatives possess a strong oxidizing potential that can damage a wide diversity of biological molecules, including the electron-rich bases of DNA, which results into single- and double-stranded breaks (Amor et al., 1998; Dizdaroglu et al., 2002; Roldan-Mona and Ariza, 2009). Hydrogen peroxide (H₂O₂) is a major ROS compound and is able to transverse cellular membranes, migrating into different compartments. This feature grants H₂O₂ not only the potential to damage a variety of cellular structures, but also to serve as a signaling molecule, allowing the activation of pathways that modulate developmental, metabolic and defense pathways (Mittler et al., 2011). One of the signaling effects of H₂O₂ is the activation of a cell division arrest by cell cycle checkpoint activation (Tsukagoshi, 2012), however, the molecular mechanisms involved remain unknown.

Cell cycle checkpoints adjust cellular proliferation to changing growth conditions, arresting it by the inhibition of the main cell cycle controllers: the heterodimeric complexes between the cyclin-dependent kinases (CDK) and the regulatory cyclins (Lee and Nurse, 1987; Norbury and Nurse, 1992). The activators of these checkpoints are the highly conserved ATAXIA TELANGIECTASIA MUTATED (ATM) and ATM AND RAD3-RELATED (ATR) kinases that are recruited in accordance with the type of DNA damage (Zhou and Elledge, 2000; Abraham, 2001; Bartek and Lukas, 2001; Kurz and Lees-Miller, 2004). ATM is activated by double-stranded breaks (DSBs); whereas ATR is activated by single-stranded breaks or stalled replication forks, causing inhibition of DNA replication. In mammals, ATM and ATR activation result in the phosphorylation of the Chk2 and Chk1 kinases, respectively. In mammals, both kinases subsequently phosphorylate p53, a critical transcription factor responsible to conduct DNA damage responses (Chaturvedi et al., 1999; Shieh et al., 2000; Chen and Sanchez, 2004; Rozan and El-Deiry, 2007). p53 seemingly appears to have no plant ortholog, although an analogous role for p53 is suggested for the plant-specific SUPPRESSOR OF GAMMA RESPONSE 1 (SOG1) transcription factor that is under direct post-transcriptional control of ATM (Yoshiyama et al., 2009; Yoshiyama et al., 2013). Another distinct feature relates to the inactivation of CDKs in response to DNA stress. CDK activity is in part controlled by its phosphorylation status at the N-terminus, determined by the interplay of the CDC25 phosphatase and the antagonistic WEE1 kinase, acting as the “on” and “off” switches of CDK activity, respectively (Francis, 2011). Whereas in mammals and budding yeast, the activation of the DNA replication checkpoint, leading to a cell cycle arrest, is predominantly achieved by the inactivation of the CDC25 phosphatase, as plant cells respond to replication stress by transcriptional induction of WEE1 (De Schutter et al., 2007). In absence of WEE1, Arabidopsis thaliana plants become hypersensitive to replication inhibitory drugs such as hydroxyurea (HU), which causes a depletion of dNTPs because of an inhibition of the ribonucleotide reductase (RNR) protein. However. WEE1-deficient plants respond similarly to control plants exposed to other types of DNA damage (De Schutter et al., 2007; Dissmeyer et al., 2009). Other, yet to be identified pathways controlling cell cycle progression under DNA stress, operating independently of WEE1, may exist.

There are several potential candidates to operate in checkpoint activation upon DNA stress mainly belonging to the family of CDK inhibitors (CKIs). CKI proteins are mostly low molecular weight proteins that inhibit cell division by their direct interaction with the CDK and/or cyclin subunit (Sherr and Roberts, 1995; De Clercq and Inzé, 2006). The first identified class of plant CKIs was the ICK/KRP (interactors of CDK/Kip-related protein) protein family comprising seven members in A. thaliana, all sharing a conserved C-terminal domain being similar to the CDK-binding domain of the animal CIP/KIP proteins (Wang et al., 1998; Wang et al., 2000; De Veylder et al., 2001). The TIC (tissue-specific inhibitors of CDK) is the most recently suggested class of CKIs (DePaoli et al., 2012) and encompasses SCI1 in tobacco, the only tissue-specific CKI reported so far (DePaoli et al., 2011). SCI1 shares no outstanding sequence similarity with the other classes of CKIs in plants, and has been suggested to connect cell cycle progression and auxin signaling in pistils (DePaoli et al., 2012). The third class of CKIs is the plant-specific SIAMESE/SIAMESE-RELATED (SIM/SMR) gene family. SIM has been identified as a cell cycle inhibitor with a role in trichome development and endocycle control (Churchman et al., 2006). Based on sequence analysis, five additional gene family members have been identified in A. thaliana, and together with EL2 from rice, been suggested to act as cell cycle inhibitors modulated either by biotic and abiotic stresses (Peres et al., 2007). Plants subjected to treatments inducing DSBs showed a rapid and strong induction of specific family members (Culligan et al., 2006; Adachi et al., 2011).

### SUMMARY OF THE DISCLOSURE

Surprisingly, it was found that three SMR genes (SMR4, SRM5 and SMR7) are transcriptionally activated by DNA damage. Even more surprisingly, the SMR5 gene encodes for a novel protein not described earlier. Cell cycle inhibitory activity was demonstrated by overexpression analysis, whereas knockout data illustrated that both SMR5 and SMR7 are essential for DNA cell cycle checkpoint activation in leaves of plants grown in the presence of HU. Remarkably, it was found that SMR induction mainly depends on ATM and SOG1, rather than ATR as would be expected for a drug that triggers replication fork defects. Correspondingly, it was demonstrated that the HU-dependent activation of SMR genes is triggered by ROS rather than replication problems, linking SMR genes with cell cycle checkpoint activation upon the occurrence of DNA damage-inducing oxidative stress.

A first aspect of the disclosure is the use of SMR5, or a homologue, orthologue or paralogue thereof, to modulate ROS signaling and/or oxidative stress response in plants. In a preferred embodiment, this use is combined with the use of SMR4 and/or SMR7. The “use of an SMR,” as used herein, comprises the use of the gene, and/or the use of the protein encoded by the gene. Preferably, the use of SMR5 is the use of a gene encoding a protein selected from the group consisting of SEQ ID NO:2, SEQ ID NO:4, and SEQ ID NO:6 of the incorporated herein Sequence Listing. In one preferred embodiment, the use of SMR5 is the use of a gene encoding a protein preferably consisting of SEQ ID NO:2. In another preferred embodiment, the use of SMR5 is the use of a gene encoding a protein preferably consisting a of a sequence selected from the group consisting of SEQ ID NO:4 and SEQ ID NO:6. “Homologues” of a protein encompass peptides, oligopeptides, polypeptides, proteins and enzymes having amino acid substitutions, deletions and/or insertions relative to the unmodified protein in question and having similar biological and functional activity as the unmodified protein from which they are derived. Orthologues and paralogues encompass evolutionary concepts used to describe the ancestral relationships of genes. Paralogues are genes within the same species that have originated through duplication of an ancestral gene; orthologues are genes from different organisms that have originated through speciation, and are also derived from a common ancestral gene.

Preferably, the use is a down-regulation of the expression of the protein, and/or the inactivation of the protein. Preferably, the down-regulation is used to improve oxidative stress tolerance in plants. “Improve” as used herein, means that the plants wherein the SMR is down-regulated have a significantly better oxidative stress resistance than the plants with the same genetic background, except for the modifications needed for the down-regulation, grown under the same conditions. Methods for down-regulation are known to the person skilled in the art, and include, but are not limited to, mutations, insertions or deletions in the gene and/or its promoter, the use of anti-sense RNA or RNAi and gene silencing methods. Methods to induce site-specific mutations in plants are known to the person skilled in the art and include Zinc-finger nucleases, transcription activator-like nucleases (TALENs) and the clustered regularly interspaced short palindromic repeat (CRISPR)/Cas-based RNA guided DNA endonucleases (Gaj et al., 2013). Inactivation of the protein can be obtained, as a non-limiting example, by the use of antigen-binding proteins directed against the protein, or by protein aggregation, as described in WO 2012/123419. The down-regulation of SMR5 can be measured by measuring the activity of its substrate (Cyclin-dependent kinase A, CDKA) as described in De Veylder et al. (1997); a higher CDKA activity points to a down-regulation of SMR5.

A “plant” as used herein may be any plant. Plants include gymnosperms and angiosperms, monocotyledons and dicotyledons, trees, fruit trees, field and vegetable crops and ornamental species. Preferably, the plant is a crop plant including, but not limited to, soybean, corn, wheat, barley and rice.

Another aspect of the disclosure is a genetically modified plant comprising an inactivated SMR5 gene and/or protein. “Inactivated,” as used herein, means that the activity of the inactivated form is significantly lower than that of the active form. “Significantly,” as used herein, means that the activity of the mutant gene or protein is at least 20% lower, preferably at least 50% lower, more preferably at least 75% lower, most preferably at least 90% lower than the wild-type gene or protein. Preferably, the activity of the gene is measured as the amount of messenger RNA. Preferably, the activity of the protein is measured as inhibition of cell division. In one preferred embodiment, the active form of the gene is encoding a protein preferably consisting of SEQ ID NO:2. In another preferred embodiment, the use of SMR5 is the use of a gene encoding a protein preferably consisting of a sequence selected from the group consisting of SEQ ID NO:4 and SEQ ID NO:6. In a preferred embodiment, the plant is a maize plant in which ZmSMRg and/or ZmSMRh are inactivated, preferably as a CRISPR/Cas knock out.

In one preferred embodiment, the gene encoding the SMR5p is disrupted. In another preferred embodiment, the gene encoding the SMR5p is silenced. In still another embodiment, the SMR5p itself is inactivated by protein aggregation.

Preferably, the genetically modified plant further comprises an inactivated SMR4 gene and/or protein, and/or an inactivated SMR7 gene and/or protein.

Still another aspect of the disclosure is a method to increase oxidative stress resistance in a plant comprising the down-regulation of SMR5p expression and/or activity. Preferably, the down-regulation is combined with the down-regulation of SMR4p expression and/or activity, and/or down-regulation of SMR7p expression and/or activity.

In one preferred embodiment, the method comprises a step wherein the plant is transformed with an RNAi construct against one or more of the SMR genes. In one preferred embodiment, the RNAi construct is placed under control of a constitutive promoter. In another preferred embodiment, the RNAi construct is placed under control of an oxidative stress-inducible promoter.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1: DNA stress meta-analysis. Venn diagram showing the overlap between transcripts induced by hydroxyurea (HU), bleomycin (Bm), and γ-radiation (γ-rays). In total, 61 genes were positively regulated in at least two DNA stress experiments, and 22 genes accumulated in all DNA stress experiments.

FIGS. 2A and 2B: Hierarchical average linkage clustering of SIM/SMR genes induced in response to different abiotic (FIG. 2A) and biotic stresses (FIG. 2B). Data comprise the SIM/SMR represented in publicly available AFFYMETRIX® ATHI microarrays obtained with the GENEVESTIGATOR® toolbox. Blue and yellow indicate down- and up-regulation, respectively, whereas black indicates no change in expression.

FIG. 3: SIM/SMR induction in response to HU. One-week-old transgenic Arabidopsis seedlings were transferred to control (−HU) medium or medium supplemented with 1 mM HU (+HU). GUS assays were performed 24 hours after transfer.

FIG. 4: SIM/SMR induction in response to Bleomycine. One-week-old transgenic Arabidopsis seedlings were transferred to control (−Bm) medium or medium supplemented with 0.3 μg/mL bleomycin (+Bm). GUS assays were performed after 24 hours after transfer.

FIG. 5: Transcriptional induction of SIM/SMR genes upon HU and bleomycin treatment. One-week-old wild-type Arabidopsis seedlings were transferred to control medium (blue), or medium supplemented with 1 mM hydroxyurea (red) or 0.3 g/mL bleomycin (green). Root tips were harvested after 24 hours for RT-PCR analysis. Expression levels in control condition were arbitrarily set to one. Data represent mean±SE (n=3).

FIG. 6: Transcriptional induction of SIM/SMR genes upon γ-irradiation. (Panels A-F) PSMR4:GUS (Panels A and D), PSMR5:GUS (Panels B and E) and PSMR7:GUS (Panels C and D) either control-treated (Panels A-C) or irradiated with 20 Gy of γ-rays (Panels D-F). GUS assays were performed 1.5 hours after irradiation.

FIG. 7: Ectopic SMR4, SMR5 and SMR7 expression inhibits cell division. Panels A-D, Four-week-old rosettes of control (Panel A), SMR4^{OE} (Panel B), SMR5^{OE} (Panel C), and SMR7^{OE} (Panel D) plants. Panels E-H, Leaf abaxial epidermal cell images of in vitro-grown 3-week-old control (Panel E), SMR4^{OE} (Panel F), SMR5^{OE} (Panel G), and SMR7^{OE} (Panel H) plants. Panels I-L, Ploidy level distribution of the first leaves of 3-week-old in vitro-grown control (Panel I), SMR4^{OE} (Panel J), SMR5^{OE} (Panel K), and SMR7^{OE} (Panel L) plants.

FIGS. 8A and 8B: Graphical representation of the SMR5 and SMR7 T-DNA insertion. FIG. 8A, Intron-exon organization of the Arabidopsis SMR5 and SMR7 genes. Black and white boxes represent coding and non-coding regions, respectively, while lines represent introns. The white triangles indicate the T-DNA insertion sites. FIG. 8B, qRT-PCR analysis on wild-type, SMR5^{KO}, SMR7^{KO}, and SMR5^{KO} SMR7^{KO} seedlings using primers specific to either SMR5 or SMR7. Expression levels in wild-type were arbitrarily set to one. Data represent mean±SE (n=3).

FIGS. 9A and 9B: SMR5 and SMR7 are required for an HU-dependent cell cycle checkpoint. Leaf size (FIG. 9A) and abaxial epidermal cell number (FIG. 9B) of the first leaves of 3-week-old plants grown on control medium (circles) or medium supplemented with 1 mM HU (squares). Data represent mean with 95% confidence interval (n=10).

FIGS. 10A and 10B: SMR5 and SMR7 expression is ATM- and SOG1-dependent. PSMR5:GUS (FIG. 10A) and PSMR7:GUS (FIG. 10B) reporter constructs introgressed into atr-2, atm-1 and sog-1 mutant backgrounds were control-treated (Ctrl), or treated with HU or bleomycin (Bm) for 24 hours.

FIGS. 11A-11C: HU triggers oxidative stress. FIG. 11A, H₂O₂ scavenging of control, HU- and 3-AT (positive control) treated plants. Error bars show SEM (n=3-4). FIG. 11B, Maximum quantum efficiency of PSII (F′v/F′m) of seedlings grown under low (LL) and high light (HL), in absence (−HU) and presence (+HU) of HU. FIG. 11C, Light microscope pictures of plants shown in FIG. 11B.

FIGS. 12A-12D: SMR5 and SMR7 are induced by oxidative stress-inducing stimuli. Relative SMR5 (FIG. 12A) and SMR7 (FIG. 12B) expression levels in wild-type (Col-0), apx1, cat2 and apx cat2 mutant plants. Expression levels in wild-type were arbitrarily set to one. Data represent mean±SE (n=3). FIG. 12C, One-week-old PSMR5:GUS and PSMR7:GUS seedlings grown under low-versus high-light conditions. FIG. 12D, Abaxial epidermal cell number of the first leaves of 3-week-old plants transferred at the age of 8 days for 48 hours to control (circles) or high light (squares) conditions. Data represent mean with 95% confidence interval (n>8).

FIG. 13: Cluster analysis of the maize SMR family with the Arabidopsis SMR5

### DETAILED DESCRIPTION

### Examples

### Materials and Methods to the Examples

### Plant Materials and Growth Conditions

The smr5 (SALK_100918) and smr7 (SALK_128496) alleles were acquired from the Arabidopsis Biological Research Center. Homozygous insertion alleles were checked by genotyping PCR using the primers listed in Table 3. The atm-1, atr-2 and sog1-1 mutants have been described previously (Garcia et al., 2003; Preuss and Britt, 2003; Culligan et al., 2004; Yoshiyama et al., 2009). Unless stated otherwise, plants of Arabidopsis thaliana (L.) Heyhn (ecotype Columbia), were grown under long-day conditions (16 hours of light, 8 hours of darkness) at 22° C. on half-strength Murashige and Skoog (MS) germination medium (Murashige and Skoog, 1962). Arabidopsis plants were treated with HU as described by Cools et al. (2011). For bleomycin treatments, five-day-old seedlings were transferred into liquid MS medium supplemented with 0.3 μg/mL bleomycin. For γ-irradiation treatments, five-day-old in vitro-grown plantlets were irradiated with γ-rays at a dose of 20 Gy. For light treatments, one-week-old seedlings were transferred to continuous high-light conditions (growth rooms kept at 22° C. with 24-hour day/0-hour night cycles and a light intensity of 300-400 μmol m⁻² s⁻¹) for 2 days, and subsequently retransferred to low-light conditions. The first leaf pair was harvested and incubated in 100% ethanol for epidermis cell drawing as described by De Veylder et al. (2001).

### DNA and RNA Manipulation

Genomic DNA was extracted from Arabidopsis leaves with the DNEASY® Plant Kit (Qiagen) and RNA was extracted from Arabidopsis tissues with the RNEASY® Mini Kit (Qiagen). After DNase treatment with the RQ1 RNase-Free DNase (Promega), cDNA was synthesized with the iScript cDNA Synthesis Kit (Bio-Rad). A quantitative RT-PCR was performed with the SYBR® Green kit (ROCHE) with 100 nM primers and 0.125 μL of RT reaction product in a total of 5μL per reaction. Reactions were run and analyzed on the LIGHTCYCLER® 480 (Roche) according to the manufacturer's instructions with the use of the following reference genes for normalization: ACTIN2 (At3g46520), EMB2386 (At1g02780), PACI (At3g22110) and RPS26C (At3g56340). Primers used for the RT-PCR are given in Table 5.

SIM/SMR promoter sequences were amplified from genomic DNA by PCR using the primers described in Table 5. The product fragments were created with the Pfu DNA Polymerase Kit (Promega, Catalog #M7745), and were cloned into a pDONR P4-Plr entry vector by BP recombination cloning and subsequently transferred into the pMK7S*NFml4GW,0 destination vector by LR cloning, resulting in a transcriptional fusion between the promoter of the SMR genes and the nlsGFP-GUS fusion gene (Karimi et al., 2007). For the overexpression constructs, the SMR coding regions were amplified using primers described in Table 5, and cloned into the pDONR221 vector by BP recombination cloning and subsequently transferred into the pK2GW7 destination vector (Kamimi et al., 2002) by LR cloning. All constructs were transferred into the Agrobacterium tumefaciens C58C1RifR strain harboring the pMP90 plasmid. The obtained Agrobacterium strains were used to generate stably transformed Arabidopsis lines with the floral dip transformation method (Clough and Bent, 1998). Transgenic plants were obtained on kanamycin-containing medium and later transferred to soil for optimal seed production. All cloning primers are listed in Table 5.

### GUS Assays

Complete seedlings or tissue cuttings were stained in multiwell plates (Falcon 3043; Becton Dickinson). GUS assays were performed as described by Beeckman and Engler (1994). Samples mounted in lactic acid were observed and photographed with a stereomicroscope (Olympus BX51 microscope) or with a differential interference contrast (DIC) microscope (Leica).

### Microscopy

For leaf measurements, first leaves were harvested at 21 days after sowing on control medium, medium supplemented with 1 mM hydroxyurea or 0.3 μg/mL bleomycin. Leaves were cleared overnight in ethanol, stored in lactic acid for microscopy, and observed with a microscopy fitted with DIC optics (Leica). The total (blade) area was determined from images digitized directly with a digital camera (Olympus BX51 microscope) mounted on a binocular (Stemi SV 11; Zeiss). From scanned drawing-tube images of the outlines of at least 30 cells of the abaxial epidermis located between 25% to 75% of the distance between the tip and the base of the leaf, halfway between the midrib and the leaf margin, the following parameters were determined: total area of all cells in the drawing and total numbers of pavement and guard cells, from which the average cell area was calculated. The total number of cells per leaf was estimated by dividing the leaf area by the average cell area. For confocal microscopy, root meristems were analyzed 2 days after transfer using a Zeiss LSM 510 Laser Scanning Microscope and the LSM Browser version 4.2 software (Zeiss). Plant material was incubated for 2 minutes in a 10 μm PI solution to stain the cell walls and was visualized with a HeNe laser through excitation at 543 nm. GFP fluorescence was detected with the 488-nm line of an Argon laser. GFP and PI were detected simultaneously by combining the settings indicated above in the sequential scanning facility of the microscope. Acquired images were quantitatively analyzed with the ImageJ v1.45s software (on the World Wide Web at rsbweb.nih.gov/ij/) and Cell-o-Tape plug-ins (French et al., 2012). Chlorophyll a fluorescence parameters were measured using the IMAGING PAM M-Series Chlorofyll Fluorescence (Walz) and associated software.

### Flow Cytometry Analysis

For flow cytometric analysis, root tip tissues were chopped with a razor blade in 300 μL of 45 mM MgCl₂, 30 mM sodium citrate, 20 mM MOPS, pH 7 (Galbraith et al., 1991). One microliter of 4,6-diamidino-2-phenylindole (DAPI) from a stock of 1 mg/mL was added to the filtered supernatant. Leaf material was chopped in 200 μL of Cystain UV Precise P Nuclei extraction buffer (Partec), supplemented with 800 μL of staining buffer. The mix was filtered through a 50-μm green filter and read by the CYFLOW® MB flow cytometer (Partec). The nuclei were analyzed with the CYFLOGIC® software.

### Catalase Assay

Plants were germinated on either control medium, medium with 1 mM HU or 6 μM 3-AT. Leaf tissue of 10 plants was ground in 200 μL extraction buffer (60 mM Tris (pH 6.9), 1 mM phenylmethylsulfonylfluoride, 10 mM DTT) on ice. The homogenate was centrifuged at 13,000 g for 15 minutes at 4° C. A total of 45 μg protein extract was mixed with potassium phosphate buffer (50 mM, pH 7.0) (Vandenabeele et al., 2004). After addition of 11.4 μL H₂O₂ (7.5%), the absorbance of the sample at 240 nm after 0 and 60 seconds was measured to determine catalase activity by H₂O₂ breakdown (Beers and Sizer, 1952; Vandenabeele et al., 2004).

### Microarray Analysis

Seeds were plated on sterilized membranes and grown under a 16-hour light/8-hour dark regime at 21° C. After 2 days of germination and 5 days of growth, the membrane was transferred to MS medium containing 0.3 μg/mL bleomycin for 24 hours. Triplicate batches of root meristem material seedlings were harvested for total RNA preparation using the RNEASY® plant mini kit (Qiagen). Each of the different root tip RNA extracts were hybridized to 12 AFFYMETRIX® Arabidopsis Gene 1.0 ST Arrays according to manufacturer's instructions at the Nucleomics Core Facility (Leuven, Belgium; World Wide Web at nucleomics.be). Raw data were processed with the RMA algorithm (Irizarry et al., 2003) using the AFFYMETRIX® Power Tools and subsequently subjected to a Significance Analysis of Microarray (SAM) analysis with “MultiExperiment Viewer 4” (MeV4) of The Institute for Genome Research (TIGR) (Tusher et al., 2001). The imputation engine was set as 10-nearest neighbor imputer and the number of permutations was 100. Expression values were obtained by log 2-transforming the average value of the normalized signal intensities of the triplicate samples. Fold changes were obtained using the expression values of the treatment relative to the control samples. Genes with Q-values<0.1 and fold change>1.5 or <0.666 were retained for further analysis.

### Microarray Meta-Analysis

Transcripts induced by bleomycin (Q-value<0.1 and fold change>1.5) were compared with different published DNA stress-related data sets. For γ-irradiation, an intersect of the genes with a significant induction (P-value<0.05, Q-value<0.1, and fold change>1.5) in 5-day-old wild-type seedlings 1.5 hours post-irradiation (100 Gy) was made of two independent experiments (Culligan et al., 2006; Yoshiyama et al., 2009). For replication stress, genes showing a significant induction (P-value (Time)<0.05, Q-value (Time)<0.1 and fold change>1.5) in 5-day-old wild-type root tips after 24 hours of 2-mM hydroxyurea treatment were selected (Cools et al., 2011). Meta-analysis of the SMR genes during various stress conditions and treatments were obtained using GENEVESTIGATOR® (Hruz et al., 2008). Using the “Response Viewer” tool, the expression profiles of genes following different stimuli were analyzed. Only biotic and abiotic stress treatments with a more than 2-fold change in the transcription level (P-value<0.01) for at least one of the SMR genes were taken into account. Fold-change values were hierarchically clustered for genes and experiments by average linkage in MeV from TIGR.

### Accession Numbers

Microarray results have been submitted to MiamExpress (on the World Wide Web at ebi.ac.uk/miamexpress), with accession E-MEXP-3977. Sequence data from this article can be found in the Arabidopsis Genome Initiative or GenBank/EMBL databases under the following accession numbers: SMR4 (At5g02220); SMR5 (At1g07500); SMR7 (At3g27630); ATM (At3g48490); ATR (At5g40820); and SOG1 (At1g25580).

### Example 1

### Meta-Analysis of DNA Stress Datasets Identifies DNA Damage-Induced SMR Genes

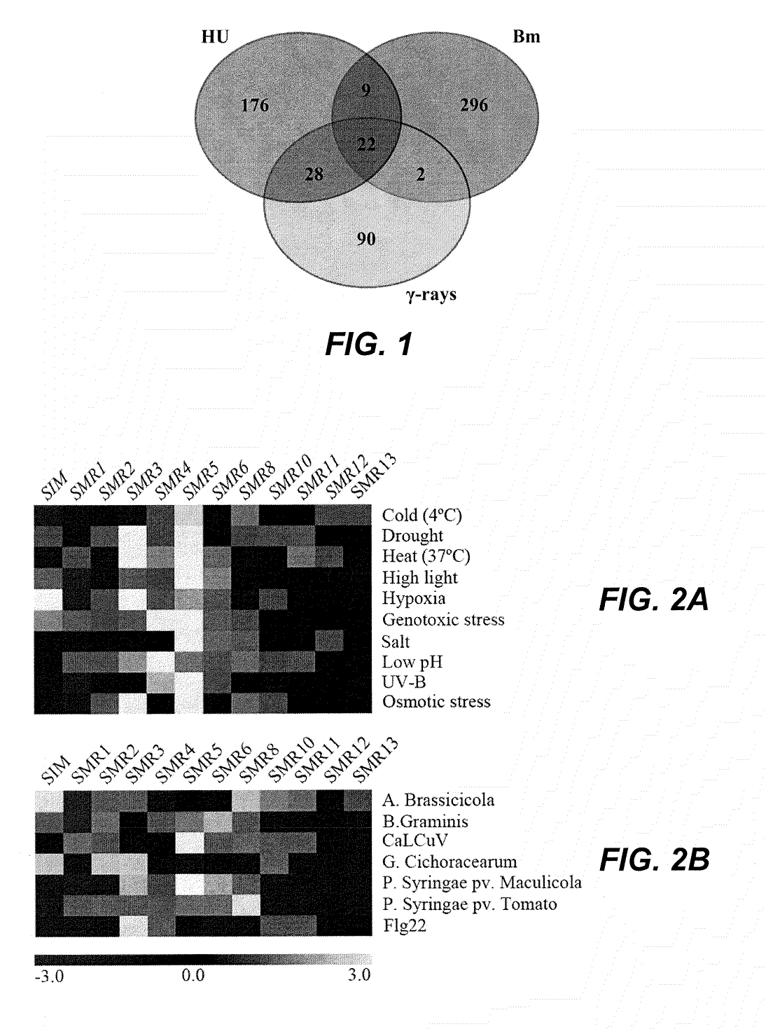

When DNA damage occurs, two global cellular responses are essential for cell survival: activation of the DNA repair machinery and delay or arrest of cell cycle progression. In recent years, gene expression inventories have been collected that focus on the transcriptional changes in response to different types of DNA stress (Culligan et al., 2006; Ricaud et al., 2007; Yoshiyama et al., 2009; Cools et al., 2010). To identify novel key signaling components that contribute to cell cycle checkpoint activation, bleomycin-induced genes were compared to those induced by HU treatment (Cools et al., 2010) and γ-radiation (Culligan et al., 2006; Yoshiyama et al., 2009). Twenty-two genes were up-regulated in all DNA stress experiments and can be considered as transcriptional hallmarks of the DNA damage response (DDR), regardless of the type of DNA stress (FIG. 1; Table 1). Within this selection, genes known to be involved in DNA stress and DNA repair are predominantly present, including PARP2, BRCA1 and RAD51. In addition, one member of the SIM/SMR gene family was recognized, being SMR5 (At1g07500). When expanding the selection by considering genes induced in at least two of the three DNA stress experiments, a total of 61 genes were identified (Table 2). Besides DDR-related genes, this expanded dataset included an additional SMR family member (SMR4; At5g02220), being expressed upon treatment with HU or γ-radiation.

### Example 2

### The SMR Gene Family Comprises 14 Family Members that Respond to Different Stresses

Previously, the existence of one SIM and five SMR genes (SMR1-SMR5) in the A. thaliana genome (Peres et al., 2007) was reported, whereas protein purification of CDK/cyclin complexes resulted in the identification of two additional family members (SMR6 and SMR8) (Van Leene et al., 2010). With the availability of new sequenced plant genomes, the Arabidopsis genome was re-examined using iterative BLAST searches for the presence of additional SMR genes, resulting in the identification of six non-annotated family members, nominated SMR7 to SMR13 (Table 3). With the GENEVESTIGATOR® toolbox (Hruz et al., 2008), the expression pattern of the twelve SIM/SMR genes represented on the AFFYMETRIX® ATHI microarray platform was analyzed in response to different biotic and abiotic stress treatments. Distinct family members were induced under various stress conditions, albeit with different specificity (FIGS. 2A and 2B). Every SMR gene appeared to be transcriptionally active under at least a number of stress conditions, with SMR5 responding to the most diverse types of abiotic stresses. In response to DNA stress (genotoxic stress and UV-B treatment), two SMR genes responded strongly, being SMR4 and SMR5, corresponding with their presence among the DNA stress genes identified by the microarray meta-analysis.

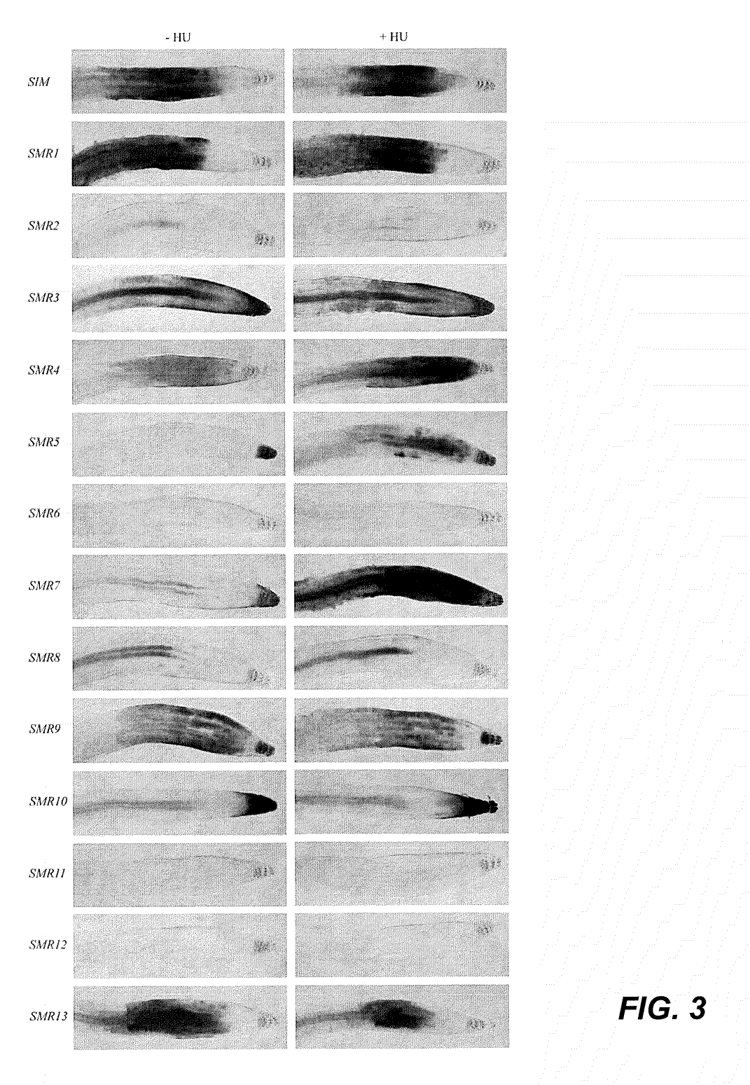

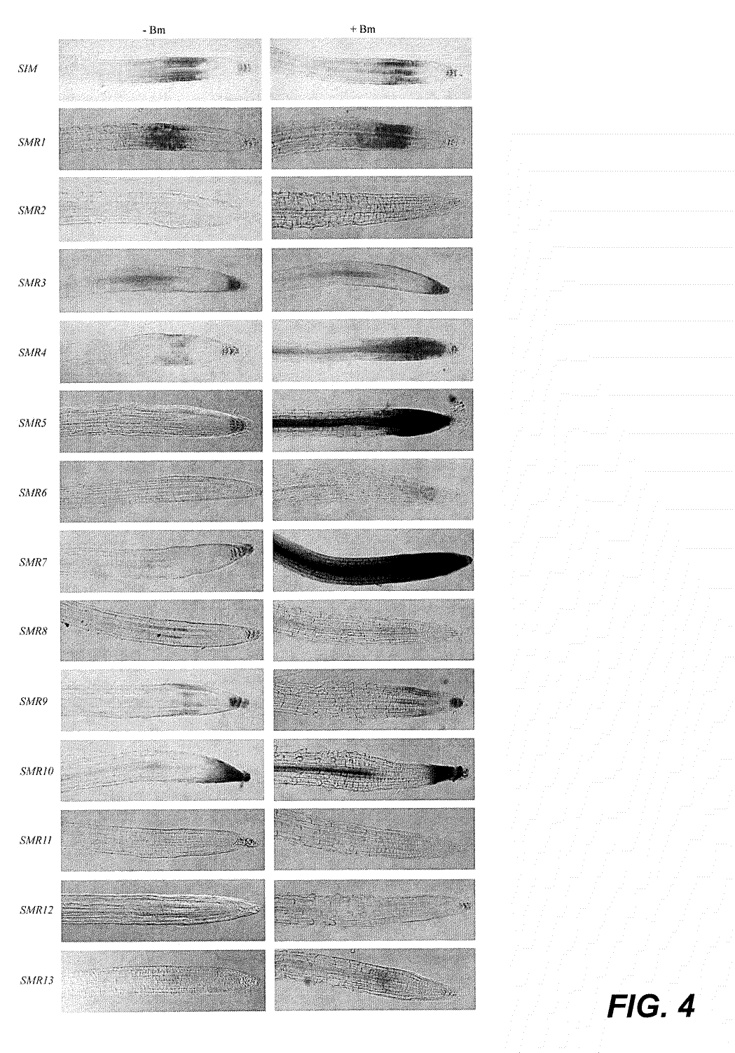

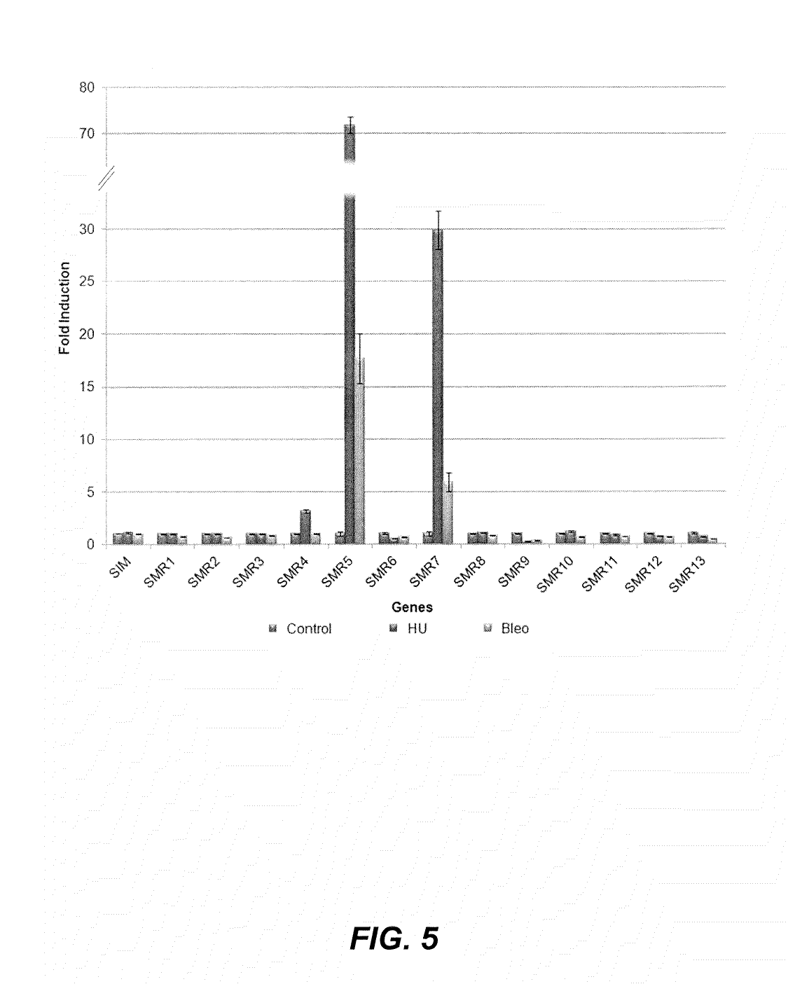

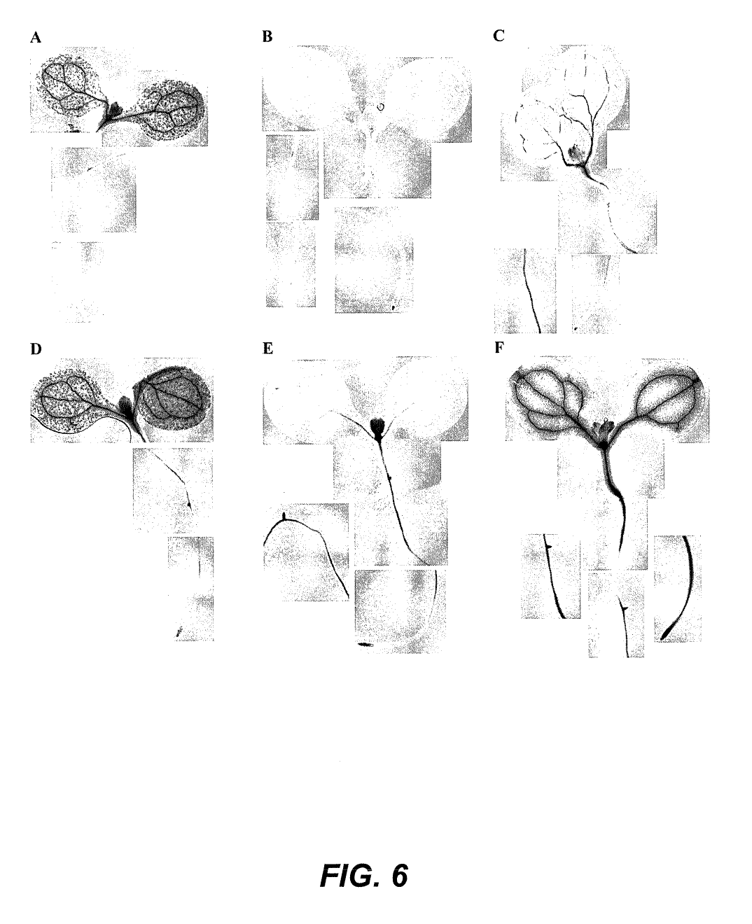

To confirm involvement of SIM/SMR genes in the genotoxic stress response, transcriptional reporter lines containing the putative upstream promoter sequences were constructed for all. After selection of representative reporter lines, one-week-old seedlings were transferred to control medium, or medium supplemented with HU (resulting into stalled replication forks) or bleomycin (causing DSBs). Focusing on the root tips revealed distinct expression patterns (FIGS. 3 and 4), with some family members being restricted to the root elongation zone (including SIM and SMR1), while others were confined to vascular tissue (e.g., SMR2 and SMR8), or columella cells (e.g., SMR5). When plants were exposed to HU, three SMR genes showed strong transcriptional induction in the root meristem, being SMR4, SMR5 and SMR7, with the latter two displaying the strongest response (FIG. 3). In the presence of bleomycin, an additional weak cell-specific induction of SMR6 was observed (FIG. 4). Transcriptional induction of SMR4, SMR5 and SMR7 by HU and bleomycin was confirmed by qRT-PCR experiments (FIG. 5). These data fit the above-described microarray analysis, with the lack of SMR7 (At3g27630) being explained by its absence on the ATHI microarray of the HU and γ-irradiation experiments, although being induced 5.68-fold in the bleomycin experiment performed using the Aragene array. Next to HU and bleomycin, transcriptional activation of SMR4, SMR5 and SMR7 was confirmed by γ-irradiation (FIG. 6).

### Example 3

### DNA Stress-Induced SMR Genes Encode Potent Cell Cycle Inhibitors

Previously, SIM had been proven to encode a potent cell cycle inhibitor, since its ectopic expression results in dwarf plants holding less cells compared to control plants (Churchman et al., 2006). To test whether the DNA stress-induced SMR genes encode proteins with cell division inhibitory activity, SMR4-, SMR5- and SMR7-overexpressing (SMR4^{OE}, SMR5^{OE} and SMR7^{OE}) plants were generated. For each gene, multiple lines with strong transcript levels were isolated, all showing a reduction in rosette size compared to wild-type plants (FIG. 7, Panels A to D). This decrease in leaf size correlated with an increase in cell size (FIG. 7, Panels E to H), indicative of a strong inhibition of cell division. Similar to SIM (Churchman et al., 2006), ectopic expression did not only inhibit cell division but also triggered an increase in the DNA content by stimulation of endoreplication (FIG. 7, Panels I to L; Table 4), likely representing a premature onset of cell differentiation. Together with the previously described biochemical interaction between SMR4 and SMR5, and CDKA;1 and D-type cyclins (Van Leene et al., 2010), it can be concluded that the DNA stress-induced SMR genes encode potent cell cycle inhibitors.

### Example 4

### SMR5 and SMR7 Control an HU-Dependent Checkpoint in Leaves

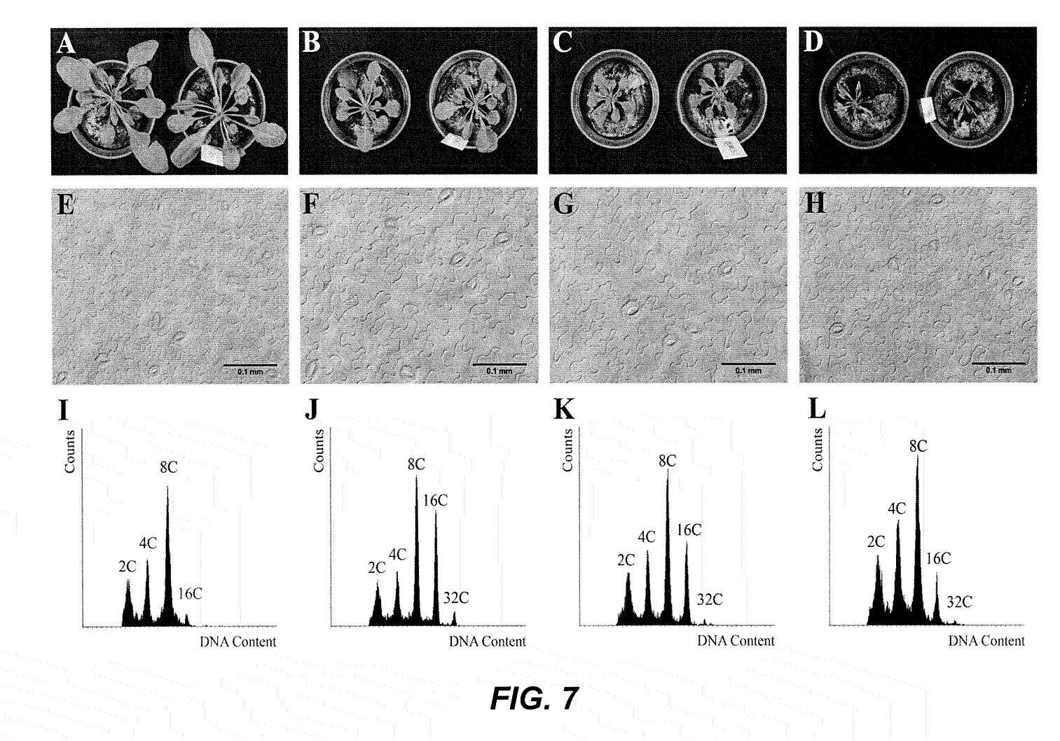

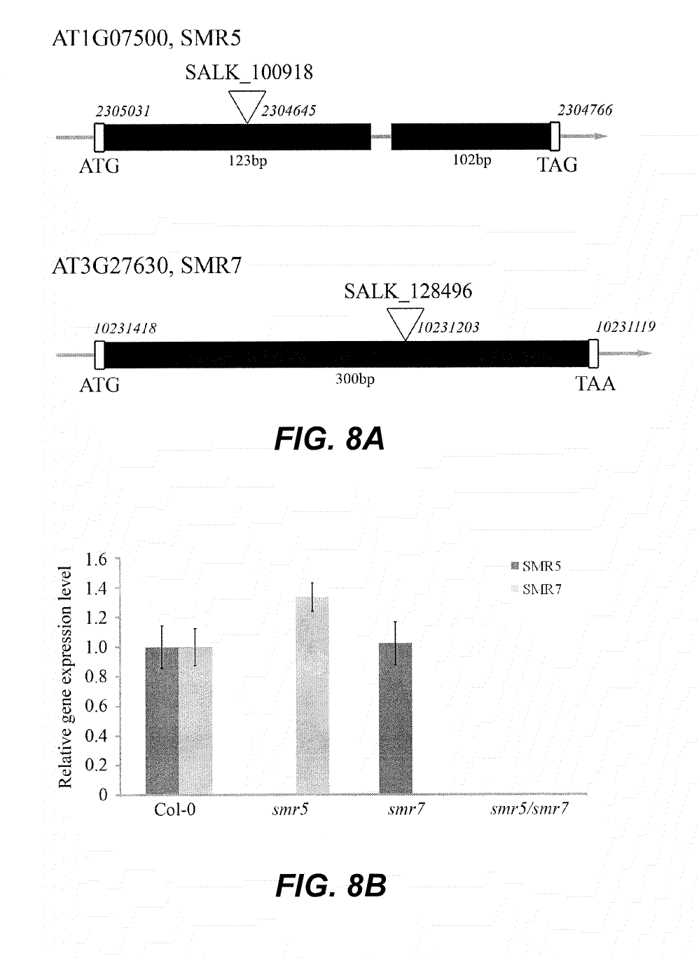

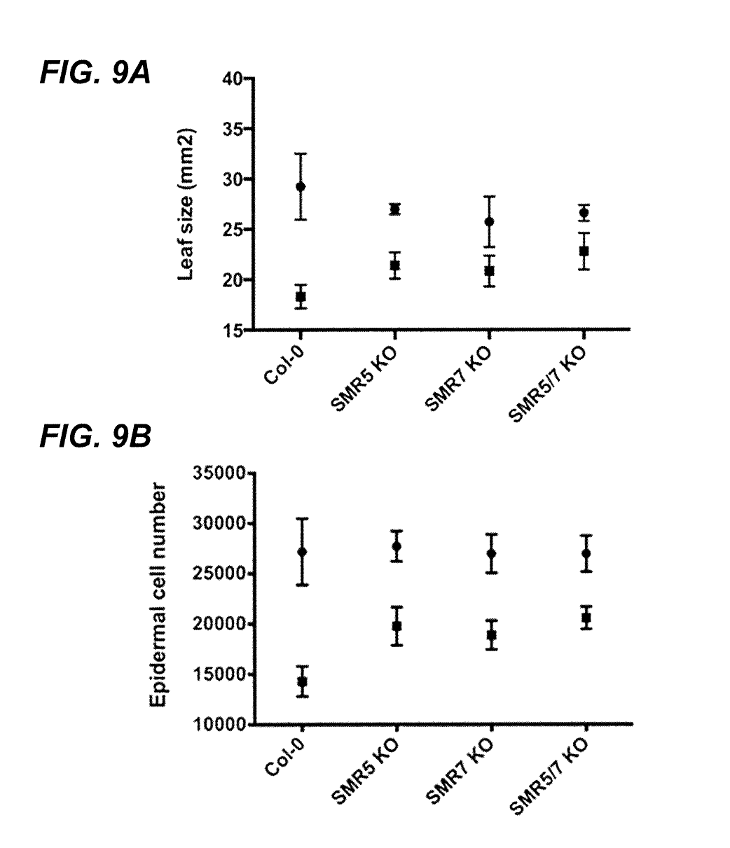

To address the role of the different SMR genes in DNA stress checkpoint control, the growth response to HU treatment of plants being knocked out for SMR5 or SMR7 (FIGS. 8A and 8B) was compared to that of control plants (Col-0). No significant difference in leaf size was observed for plants grown under standard conditions. In contrast, when comparing plants grown for 3 weeks in the presence of HU, the size of the SMR5^{KO} and SMR7^{KO} leaves was significantly bigger than that of the control plants (FIG. 9A). This difference was attributed to a difference in cell number. Control plants responded to the HU treatment with a 47% reduction in epidermal cell number, reflecting an activation of a stringent cell cycle checkpoint. In contrast, in SMR5^{KO} and SMR7^{KO} plants, this reduction was restricted to 29% and 30%, respectively (FIG. 9B). Within the SMR5^{KO} SMR7^{KO} double mutant, the reduction in leaf size and cell number was even less (FIGS. 9A and 9B), suggesting that both inhibitors contribute to the cell cycle arrest observed in the control plants by checkpoint activation upon HU stress. Unfortunately, a similar role of SMR4 could not be tested due to the lack of an available knockout.

### Example 5

### SMR5 and SMR7 Expression is Triggered by Oxidative Stress

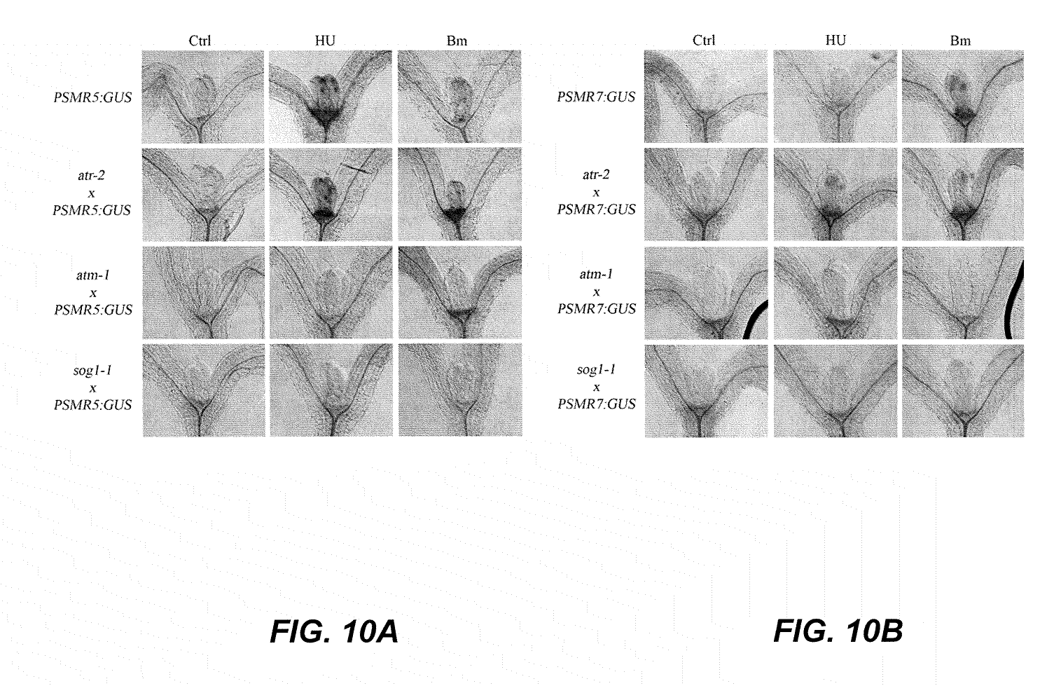

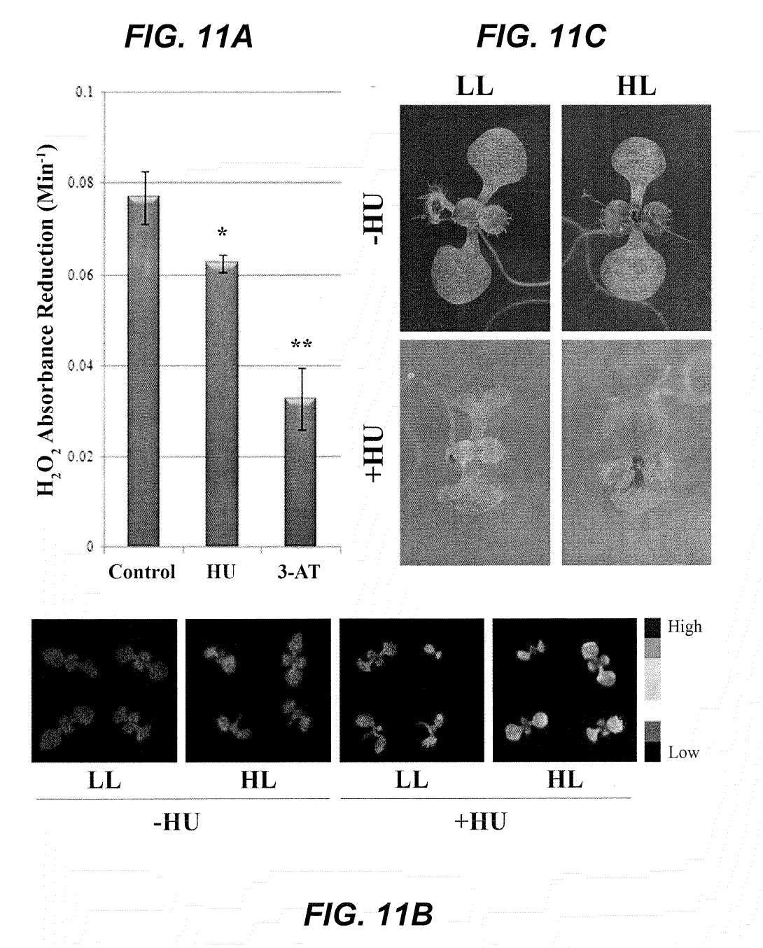

Because of the observed role of the SMR5 and SMR7 genes in DNA stress checkpoint control, the dependence of their expression on the ATM and ATR signaling kinases and the SOG1 transcription factor was analyzed by introducing the SMR5 and SMR7 GUS reporter lines into the atr-2, atm-1 and sog1-1 mutant backgrounds. Both genes were induced in the proliferating leaf upon HU and bleomycin treatment (FIGS. 10A and 10B). Moreover, as would be expected for a DSB-inducing agent, the transcriptional activation of SMR5 and SMR7 by bleomycin depended on ATM and SOG1. Surprisingly, the same pattern was observed for HU, whereas one would expect that SMR5/SMR7 induction after arrest of the replication fork would rely on ATR-dependent signaling. These data indicate that the HU-dependent activation of the SMR5 and SMR7 genes might be caused by a genotoxic effect of HU being unrelated to replication stress induced by the depletion of dNTPs. A recent study demonstrated that HU directly inhibits catalase-mediated H₂O₂ decomposition (Juul et al., 2010). Analogously, in combination with H₂O₂, HU has been demonstrated to act as a suicide inhibitor of ascorbate peroxidase (Chen and Asada, 1990). Combined, both mechanisms are likely responsible for an increase in the cellular H₂O₂ concentration, which might trigger DNA damage and consequently transcriptional induction of the SMR5 and SMR7 genes. Indeed, extracts of control plants treated with HU displayed a reduced H₂O₂ decomposition rate (FIG. 11A). As catalase and ascorbate peroxidase activity are essential for the scavenging of H₂O₂ that is generated upon high-light exposure, the effects of HU treatment on photosystem II (PSII) efficiency in one-week-old seedlings was subsequently tested after transfer from low- to high-light conditions. As illustrated in FIG. 11B, transfer for 48 hours to high light resulted in a decrease of maximum quantum efficiency of PSII (F′v/F′m). In the presence of HU, the F′v/F′m decrease was even more pronounced, which again corroborates the idea that HU might interfere with H₂O₂ scavenging. Macroscopically, plants grown in the presence of HU accumulated anthocyanins in the young leaf tissue within 48 hours after transfer, whereas plants grown on control medium showed no effect of the transfer to high light (FIG. 11C).

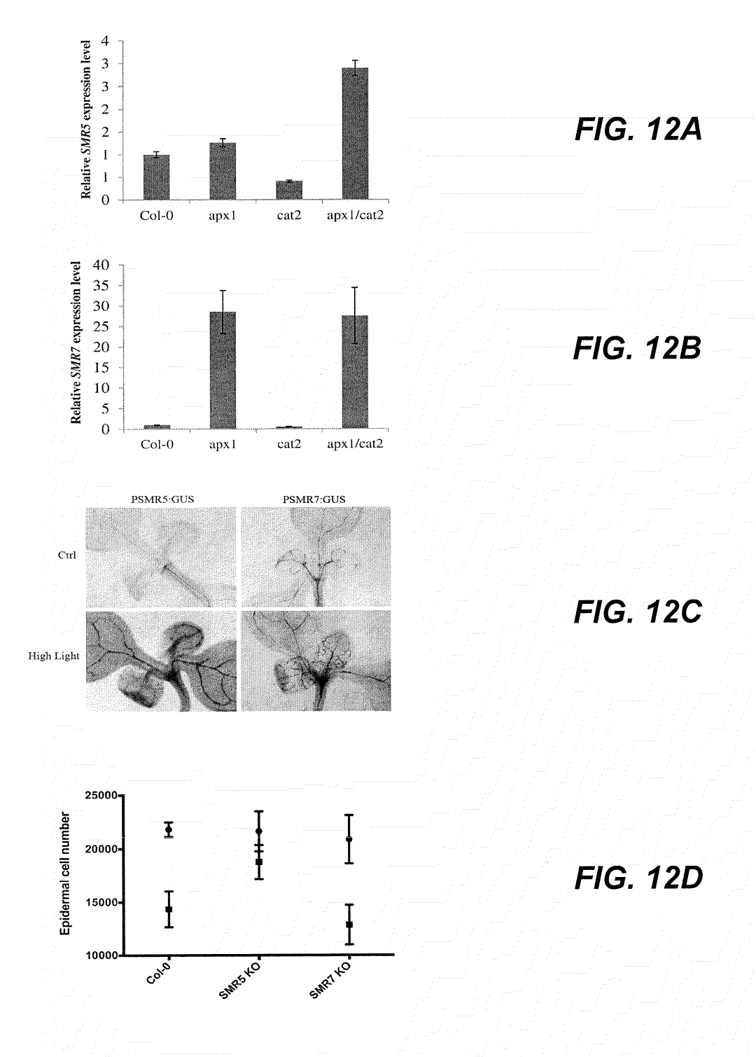

To examine whether an increase in H₂O₂ might trigger expression of SMR genes, SMR5 and SMR7 expression levels were analyzed in plants that are knockout for CAT2 and/or APX1, encoding two enzymes important for the scavenging of H₂O₂. SMR5 expression levels were clearly induced in the apx1 cat2 double mutant, whereas SMR7 transcriptional activation was observed in the apx1 knockout and apx1 cat2 double mutant (FIG. 12A). Analogously, plants grown for two days under high light conditions displayed PSMR5:GUS and SMR7:GUS induction in proliferating leaves (FIG. 12B). To examine whether this transcriptional induction contributed to a high light-induced cell cycle checkpoint, the epidermal cell numbers were measured in mature first leaves of control (Col-0), SMR5^{KO} and SMR7^{KO} plants that were transferred for two days to high light condition at the moment that their leaves were proliferating. This high light treatment resulted into a 34% and 38% reduction in cell number in control and SMR7^{KO} plants, respectively (FIG. 12C). In contrast, SMR5^{K0} plants displayed only a 13% reduction in cell number, illustrating that SMR5 is essential to activate a high light-dependent cell cycle checkpoint.

### Example 6

### Identification of Maize SMR5 Orthologues

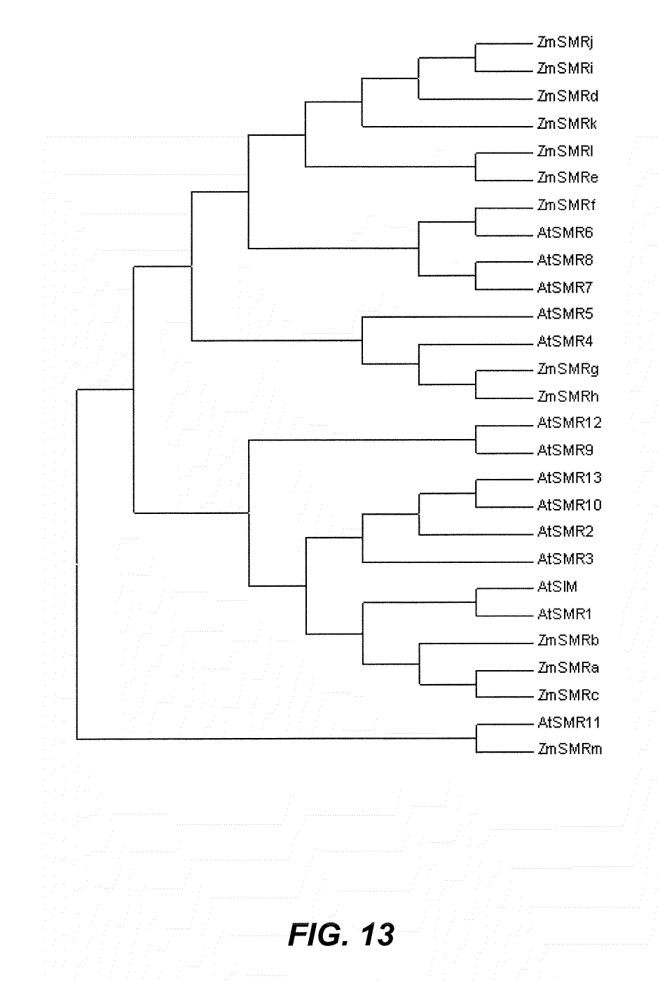

Sequences of the Arabidopsis and maize SMR proteins were aligned and subsequently clustered. The maize proteins ZmSMRg and ZmSMRh were identified as the closest orthologues of Arabidopsis SMR5. The coding sequence is given in SEQ ID NO:3 (ZmSMRg) and SEQ ID NO:5 (ZmSMRh). The results are given in FIG. 13.

The transcriptional induction of the maize SMR genes after HU treatment was measured using qRT-PCR analysis, similar as described for Arabidopsis, and both genes show a strong up-regulation upon HU treatment, both in root tips and in leaves.

Detailed expression analysis of both the ZmSMRg gene and the ZmSMRh gene is carried out using promoter-GUS fusions, transformed into maize. These transformed plants are tested under a variety of stresses including, but not limited to, drought, high light, cold, heat, hydroxyurea and bleomycin treatment.

### Example 7

### Knock Out Mutants in Maize

The ZmSMRg gene and the ZmSMRh gene are knocked out using the CRISPR-Cas technology, generating single and double knock out mutants. These knock out mutants are submitted to oxidative stress as described for Arabidopsis, and the mutants show a significant protection against oxidative stress, when compared to the wild-type grown under the same conditions.

TABLE 1
Overview of the transcriptionally induced core DNA damage
genes

| | | HU | γ-rays - | γ-rays - | |
|---|---|---|---|---|---|
| AGI locus | Annotation | 24 h/0 h^{a} | 1^{b} | 2^{c} | Bleomycin |
| AT4G21070 | Breast cancer susceptibility1 | 10.375 | 581.570 | 57.803 | 2.386 |
| AT5G60250 | Zinc finger (C3HC4-type RING finger) | 8.907 | 34.918 | 40.000 | 2.352 |
| | family protein | | | | |
| AT1G07500 | Siamese-related 5 | 7.863 | 38.160 | 35.842 | 1.595 |
| AT4G02390 | Poly(ADP-ribose) polymerase | 7.701 | 131.865 | 59.172 | 2.663 |
| AT3G07800 | Thymidine kinase | 7.160 | 46.179 | 20.492 | 2.759 |
| AT5G03780 | TRF-like 10 | 7.111 | 108.316 | 23.474 | 1.600 |
| AT5G64060 | NAC domain containing protein 103 | 5.579 | 28.086 | 13.755 | 2.153 |
| AT2G18600 | Ubiquitin-conjugating enzyme family | 5.521 | 21.462 | 11.481 | 1.972 |
| | protein | | | | |
| AT4G22960 | Unknown function (DUF544) | 5.315 | 36.380 | 14.451 | 2.282 |
| AT5G48720 | X-ray induced transcript 1 | 5.296 | 285.166 | 65.789 | 2.228 |
| AT5G24280 | Gamma-irradiation and mitomycin c | 4.823 | 108.578 | 42.918 | 2.584 |
| | induced 1 | | | | |
| AT5G20850 | RAS associated with diabetes protein | 4.643 | 186.456 | 31.250 | 1.765 |
| | 51 | | | | |
| AT3G27060 | Ferritin/ribonucleotide reductase-like | 4.595 | 37.351 | 8.741 | 1.970 |
| | family protein | | | | |
| AT2G46610 | RNA-binding (RRM/RBD/RNP motifs) | 3.593 | 19.913 | 7.331 | 1.546 |
| | family protein | | | | |
| AT5G40840 | Rad21/Rec8-like family protein | 3.375 | 113.919 | 27.473 | 1.692 |
| AT1G13330 | Hop2 homolog | 2.949 | 17.349 | 13.495 | 1.580 |
| AT5G66130 | RADIATION SENSITIVE 17 | 2.888 | 30.411 | 10.384 | 1.627 |
| AT1G17460 | TRF-like 3 | 2.378 | 18.925 | 10.661 | 1.681 |
| AT2G45460 | SMAD/FHA domain-containing protein | 2.378 | 45.673 | 21.053 | 1.575 |
| AT5G49480 | Ca2+-binding protein 1 | 1.952 | 15.106 | 5.851 | 1.580 |
| AT3G25250 | AGC (cAMP-dependent, cGMP- | 1.853 | 12.995 | 17.794 | 1.517 |
| | dependent and protein kinase C) kinase | | | | |
| | family protein | | | | |
| AT5G55490 | Gamete expressed protein 1 | 1.670 | 71.489 | 34.722 | 2.407 |

^{a}According to Cools et al., 2011
^{b}According to Culligan et al., 2006
^{c}According to Yoshiyama et al., 2009

TABLE 2
Meta-analysis of genes induced in multiple DNA damage experiments.

| | | q- | p- | | q- | p- | | q- | p- | | q- | |
|---|---|---|---|---|---|---|---|---|---|---|---|---|
| | | value | value | | value | value | | value | value | | value | |
| | | (HU - | (HU - | HU | (γ-rays - | (γ-rays - | γ-rays - | (γ-rays - | (γ-rays - | γ-rays - | Bleo- | Bleo- |
| Locus | Description | Time)^{a} | Time)^{a} | 24 h/0 h^{a} | 1)^{b} | 1)^{b} | 1^{b} | 2)^{c} | 2)^{c} | 2^{c} | mycin | mycin |
| Significantly | | | | | | | | | | | | |
| Induced by | | | | | | | | | | | | |
| HU, BM and | | | | | | | | | | | | |
| gammarays | | | | | | | | | | | | |
| AT4G21070 | breast cancer | 0.018 | 0.001 | 10.375 | 0.000 | 0.000 | 581.570 | 0.000 | 0.000 | 57.803 | 0.000 | 2.386 |
| | susceptibility1 | | | | | | | | | | | |
| AT5G60250 | zinc finger | 0.000 | 0.000 | 8.907 | 0.001 | 0.000 | 34.918 | 0.000 | 0.000 | 40.000 | 0.000 | 2.352 |
| | (C3HC4-type | | | | | | | | | | | |
| | RING finger) | | | | | | | | | | | |
| | family protein | | | | | | | | | | | |
| AT1G07500 | unknown | 0.000 | 0.000 | 7.863 | 0.003 | 0.000 | 38.160 | 0.000 | 0.001 | 35.842 | 0.000 | 1.595 |
| | protein; Has 4 | | | | | | | | | | | |
| | Blast hits to 4 | | | | | | | | | | | |
| | proteins in 3 | | | | | | | | | | | |
| | species: | | | | | | | | | | | |
| | Archae - 0; | | | | | | | | | | | |
| | Bacteria - 0; | | | | | | | | | | | |
| | Metazoa - 0; | | | | | | | | | | | |
| | Fungi - 0; | | | | | | | | | | | |
| | Plants - 4; | | | | | | | | | | | |
| | Viruses - 0; | | | | | | | | | | | |
| | Other | | | | | | | | | | | |
| | Eukaryotes - 0 | | | | | | | | | | | |
| | (source: | | | | | | | | | | | |
| | NCBI BLink). | | | | | | | | | | | |
| AT4G02390 | poly(ADP- | 0.000 | 0.000 | 7.701 | 0.001 | 0.000 | 131.865 | 0.000 | 0.000 | 59.172 | 0.000 | 2.663 |
| | ribose) | | | | | | | | | | | |
| | polymerase | | | | | | | | | | | |
| AT3G07800 | Thymidine | 0.033 | 0.002 | 7.160 | 0.000 | 0.000 | 46.179 | 0.000 | 0.004 | 20.492 | 0.000 | 2.759 |
| | kinase | | | | | | | | | | | |
| AT5G03780 | TRF-like 10 | 0..018 | 0.001 | 7.111 | 0.005 | 0.000 | 108.316 | 0.000 | 0.003 | 23.474 | 0.036 | 1.600 |
| AT5G64060 | NAC domain | 0.014 | 0.000 | 5.579 | 0.004 | 0.000 | 28.086 | 0.000 | 0.008 | 13.755 | 0.002 | 2.153 |
| | containing | | | | | | | | | | | |
| | protein 103 | | | | | | | | | | | |
| AT2G18600 | Ubiquitin- | 0.009 | 0.000 | 5.521 | 0.004 | 0.000 | 21.462 | 0.000 | 0.014 | 11.481 | 0.004 | 1.972 |
| | conjugating | | | | | | | | | | | |
| | enzyme | | | | | | | | | | | |
| | family protein | | | | | | | | | | | |
| AT4G22960 | Protein of | 0.012 | 0.000 | 5.315 | 0.009 | 0.000 | 36.380 | 0.000 | 0.009 | 14.451 | 0.000 | 2.282 |
| | unknown | | | | | | | | | | | |
| | function | | | | | | | | | | | |
| | (DUF544) | | | | | | | | | | | |
| AT5G48720 | x-ray induced | 0.048 | 0.003 | 5.296 | 0.004 | 0.000 | 285.166 | 0.000 | 0.000 | 65.789 | 0.000 | 2.228 |
| | transcript 1 | | | | | | | | | | | |
| AT5G24280 | gamma- | 0.026 | 0.001 | 4.823 | 0.009 | 0.000 | 108.578 | 0.000 | 0.000 | 42.918 | 0.000 | 2.584 |
| | irradiation and | | | | | | | | | | | |
| | mitomycin c | | | | | | | | | | | |
| | induced 1 | | | | | | | | | | | |
| AT5G20850 | RAS | 0.031 | 0.002 | 4.643 | 0.002 | 0.000 | 186.456 | 0.000 | 0.001 | 31.250 | 0.000 | 1.765 |
| | associated | | | | | | | | | | | |
| | with diabetes | | | | | | | | | | | |
| | protein 51 | | | | | | | | | | | |
| AT3G27060 | Ferritin/ribonucleotide | 0.012 | 0.000 | 4.595 | 0.001 | 0.000 | 37.351 | 0.000 | 0.018 | 8.741 | 0.000 | 1.970 |
| | reductase-like | | | | | | | | | | | |
| | family protein | | | | | | | | | | | |
| AT2G46610 | RNA-binding | 0.027 | 0.001 | 3.593 | 0.002 | 0.000 | 19.913 | 0.000 | 0.021 | 7.331 | 0.021 | 1.546 |
| | (RRM/RBD/RNP | | | | | | | | | | | |
| | motifs) | | | | | | | | | | | |
| | family protein | | | | | | | | | | | |
| AT5G40840 | Rad21/Rec8- | 0.052 | 0.004 | 3.375 | 0.005 | 0.000 | 113.919 | 0.000 | 0.002 | 27.473 | 0.002 | 1.692 |
| | like family | | | | | | | | | | | |
| | protein | | | | | | | | | | | |
| AT1G13330 | Arabidopsis | 0.014 | 0.000 | 2.949 | 0.019 | 0.000 | 17.349 | 0.000 | 0.009 | 13.495 | 0.046 | 1.580 |
| | Hop2 | | | | | | | | | | | |
| | homolog | | | | | | | | | | | |
| AT5G66130 | Radiation | 0.009 | 0.000 | 2.888 | 0.003 | 0.000 | 30.411 | 0.000 | 0.015 | 10.384 | 0.002 | 1.627 |
| | Sensitive 17 | | | | | | | | | | | |
| AT1G17460 | TRF-like 3 | 0.052 | 0.004 | 2.378 | 0.000 | 0.000 | 18.925 | 0.000 | 0.015 | 10.661 | 0.007 | 1.681 |
| AT2G45460 | SMAD/FHA | 0.012 | 0.000 | 2.378 | 0.000 | 0.000 | 45.673 | 0.000 | 0.004 | 21.053 | 0.010 | 1.575 |
| | domain- | | | | | | | | | | | |
| | containing | | | | | | | | | | | |
| | protein | | | | | | | | | | | |
| AT5G49480 | Ca2+-binding | 0.021 | 0.001 | 1.952 | 0.002 | 0.000 | 15.106 | 0.000 | 0.026 | 5.851 | 0.010 | 1.580 |
| | protein 1 | | | | | | | | | | | |
| AT3G25250 | AGC (cAMP- | 0.014 | 0.000 | 1.853 | 0.003 | 0.000 | 12.995 | 0.000 | 0.004 | 17.794 | 0.035 | 1.517 |
| | dependent, | | | | | | | | | | | |
| | cGMP- | | | | | | | | | | | |
| | dependent and | | | | | | | | | | | |
| | protein kinase | | | | | | | | | | | |
| | C) kinase | | | | | | | | | | | |
| | family protein | | | | | | | | | | | |
| AT5G55490 | gamete | 0.034 | 0.002 | 1.670 | 0.000 | 0.000 | 71.489 | 0.000 | 0.001 | 34.722 | 0.000 | 2.407 |
| | expressed | | | | | | | | | | | |
| | protein 1 | | | | | | | | | | | |
| Significantly | | | | | | | | | | | | |
| induced by | | | | | | | | | | | | |
| HU and | | | | | | | | | | | | |
| gamma rays | | | | | | | | | | | | |
| AT4G28950 | RHO-related | 0.021 | 0.001 | 9.680 | 0.000 | 0.000 | 36.081 | 0.000 | 0.008 | 13.569 | | |
| | protein from | | | | | | | | | | | |
| | plants 9 | | | | | | | | | | | |
| AT3G45730 | unknown | 0.034 | 0.002 | 5.637 | 0.000 | 0.000 | 46.290 | 0.000 | 0.009 | 14.286 | | |
| | protein; Has 3 | | | | | | | | | | | |
| | Blast hits to 3 | | | | | | | | | | | |
| | proteins in 1 | | | | | | | | | | | |
| | species: | | | | | | | | | | | |
| | Archae - 0; | | | | | | | | | | | |
| | Bacteria - 0; | | | | | | | | | | | |
| | Metazoa - 0; | | | | | | | | | | | |
| | Fungi - 0; | | | | | | | | | | | |
| | Plants - 3; | | | | | | | | | | | |
| | Viruses - 0; | | | | | | | | | | | |
| | Other | | | | | | | | | | | |
| | Eukaryotes - 0 | | | | | | | | | | | |
| | (source: | | | | | | | | | | | |
| | NCBI BLink). | | | | | | | | | | | |
| AT5G11460 | Protein of | 0.006 | 0.000 | 5.483 | 0.003 | 0.000 | 41.596 | 0.000 | 0.005 | 16.863 | | |
| | unknown | | | | | | | | | | | |
| | function | | | | | | | | | | | |
| | (DUF581) | | | | | | | | | | | |
| AT5G02220 | unknown | 0.023 | 0.001 | 4.500 | 0.001 | 0.000 | 45.759 | 0.000 | 0.004 | 20.534 | | |
| | protein; Has | | | | | | | | | | | |
| | 30201 Blast | | | | | | | | | | | |
| | hits to 17322 | | | | | | | | | | | |
| | proteins in | | | | | | | | | | | |
| | 780 species: | | | | | | | | | | | |
| | Archae - 12; | | | | | | | | | | | |
| | Bacteria - 1396; | | | | | | | | | | | |
| | Metazoa - 17338; | | | | | | | | | | | |
| | Fungi - 3422; | | | | | | | | | | | |
| | Plants - 5037; | | | | | | | | | | | |
| | Viruses - 0; | | | | | | | | | | | |
| | Other | | | | | | | | | | | |
| | Eukaryotes - 2996 | | | | | | | | | | | |
| | (source: | | | | | | | | | | | |
| | NCBI BLink). | | | | | | | | | | | |
| AT2G47680 | zinc finger | 0.031 | 0.002 | 3.422 | 0.022 | 0.000 | 50.849 | 0.000 | 0.004 | 17.513 | | |
| | (CCCH type) | | | | | | | | | | | |
| | helicase | | | | | | | | | | | |
| | family protein | | | | | | | | | | | |
| AT4G29170 | Mnd1 family | 0.060 | 0.005 | 2.898 | 0.000 | 0.000 | 40.733 | 0.000 | 0.006 | 16.694 | | |
| | protein | | | | | | | | | | | |
| AT5G06190 | unknown | 0.012 | 0.000 | 2.878 | 0.008 | 0.007 | 3.757 | 0.001 | 0.092 | 2.690 | | |
| | protein; BEST | | | | | | | | | | | |
| | Arabidopsis | | | | | | | | | | | |
| | thaliana | | | | | | | | | | | |
| | protein match | | | | | | | | | | | |
| | is: unknown | | | | | | | | | | | |
| | protein | | | | | | | | | | | |
| | (TAIR: AT3G58540.1); | | | | | | | | | | | |
| | Has 30201 Blast | | | | | | | | | | | |
| | hits to 17322 | | | | | | | | | | | |
| | proteins in | | | | | | | | | | | |
| | 780 species: | | | | | | | | | | | |
| | Archae - 12; | | | | | | | | | | | |
| | Bacteria - 1396; | | | | | | | | | | | |
| | Metazoa - 17338; | | | | | | | | | | | |
| | Fungi - 3422; | | | | | | | | | | | |
| | Plants - 5037; | | | | | | | | | | | |
| | Viruses - 0; | | | | | | | | | | | |
| | Other | | | | | | | | | | | |
| | Eukaryote | | | | | | | | | | | |
| AT5G67460 | O-Glycosyl | 0.031 | 0.002 | 2.799 | 0.005 | 0.000 | 18.032 | 0.000 | 0.004 | 17.271 | | |
| | hydrolases | | | | | | | | | | | |
| | family 17 | | | | | | | | | | | |
| | protein | | | | | | | | | | | |
| AT4G35740 | DEAD/DEAH | 0.037 | 0.002 | 2.594 | 0.002 | 0.000 | 21.434 | 0.000 | 0.021 | 7.037 | | |
| | box RNA | | | | | | | | | | | |
| | helicase | | | | | | | | | | | |
| | family protein | | | | | | | | | | | |
| AT2G21790 | ribonucleotide | 0.045 | 0.003 | 2.514 | 0.000 | 0.000 | 13.702 | 0.000 | 0.034 | 4.948 | | |
| | reductase 1 | | | | | | | | | | | |
| | SMAD/FHA | | | | | | | | | | | |
| AT3G02400 | domain- | 0.052 | 0.004 | 2.479 | 0.025 | 0.002 | 9.474 | 0.000 | 0.022 | 6.649 | | |
| | containing | | | | | | | | | | | |
| | protein | | | | | | | | | | | |
| AT2G31320 | poly(ADP- | 0.020 | 0.001 | 2.445 | 0.001 | 0.000 | 39.238 | 0.000 | 0.015 | 9.970 | | |
| | ribose) | | | | | | | | | | | |
| | polymerase 2 | | | | | | | | | | | |
| AT3G42860 | zinc knuckle | 0.039 | 0.002 | 2.445 | 0.001 | 0.000 | 30.770 | 0.000 | 0.010 | 13.351 | | |
| | (CCHC-type) | | | | | | | | | | | |
| | family protein | | | | | | | | | | | |
| AT1G09815 | polymerase | 0.026 | 0.001 | 2.354 | 0.000 | 0.000 | 19.771 | 0.000 | 0.021 | 7.310 | | |
| | delta 4 | | | | | | | | | | | |
| AT3G20490 | unknown | 0.043 | 0.003 | 2.313 | 0.003 | 0.000 | 17.593 | 0.000 | 0.029 | 5.291 | | |
| | protein; Has | | | | | | | | | | | |
| | 754 Blast hits | | | | | | | | | | | |
| | to 165 | | | | | | | | | | | |
| | proteins in 64 | | | | | | | | | | | |
| | species: | | | | | | | | | | | |
| | Archae - 0; | | | | | | | | | | | |
| | Bacteria - 48; | | | | | | | | | | | |
| | Metazoa - 26; | | | | | | | | | | | |
| | Fungi - 25; | | | | | | | | | | | |
| | Plants - 36; | | | | | | | | | | | |
| | Viruses - 0; | | | | | | | | | | | |
| | Other | | | | | | | | | | | |
| | Eukaryotes - 619 | | | | | | | | | | | |
| | (source: | | | | | | | | | | | |
| | NCBI BLink). | | | | | | | | | | | |
| AT4G19130 | Replication | 0.093 | 0.010 | 2.305 | 0.010 | 0.000 | 59.037 | 0.000 | 0.010 | 13.089 | | |
| | factor-A | | | | | | | | | | | |
| | protein 1- | | | | | | | | | | | |
| | related | | | | | | | | | | | |
| AT2G30360 | SOS3- | 0.033 | 0.002 | 2.274 | 0.004 | 0.000 | 11.137 | 0.000 | 0.017 | 9.346 | | |
| | interacting | | | | | | | | | | | |
| | protein 4 | | | | | | | | | | | |
| AT3G12510 | MADS-box | 0.006 | 0.000 | 2.266 | 0.001 | 0.000 | 17.935 | 0.000 | 0.029 | 5.426 | | |
| | family protein | | | | | | | | | | | |
| AT1G12020 | unknown | 0.030 | 0.001 | 1.873 | 0.006 | 0.000 | 8.806 | 0.001 | 0.080 | 2.976 | | |
| | protein; BEST | | | | | | | | | | | |
| | Arabidopsis | | | | | | | | | | | |
| | thaliana | | | | | | | | | | | |
| | protein match | | | | | | | | | | | |
| | is: unknown | | | | | | | | | | | |
| | protein | | | | | | | | | | | |
| | (TA1R: AT1G62422.1); | | | | | | | | | | | |
| | Has 89 Blast hits | | | | | | | | | | | |
| | to 88 proteins | | | | | | | | | | | |
| | in 16 species: | | | | | | | | | | | |
| | Archae - 0; | | | | | | | | | | | |
| | Bacteria - 0; | | | | | | | | | | | |
| | Metazoa - 0; | | | | | | | | | | | |
| | Fungi - 0; | | | | | | | | | | | |
| | Plants - 87; | | | | | | | | | | | |
| | Viruses - 0; | | | | | | | | | | | |
| | Other | | | | | | | | | | | |
| | Eukaryotes - 2 | | | | | | | | | | | |
| | (source: NCBI | | | | | | | | | | | |
| AT1G31280 | Argonaute | 0.014 | 0.000 | 1.866 | 0.002 | 0.000 | 24.264 | 0.000 | 0.017 | 9.302 | | |
| | family protein | | | | | | | | | | | |
| AT1G59660 | Nucleoporin | 0.033 | 0.002 | 1.860 | 0.014 | 0.000 | 15.946 | 0.000 | 0.013 | 11.933 | | |
| | autopeptidase | | | | | | | | | | | |
| AT3G15240 | Serine/threonine- | 0.027 | 0.001 | 1.790 | 0.016 | 0.001 | 6.471 | 0.001 | 0.060 | 3.552 | | |
| | protein | | | | | | | | | | | |
| | kinase WNK | | | | | | | | | | | |
| | (With No | | | | | | | | | | | |
| | Lysine)- | | | | | | | | | | | |
| | related | | | | | | | | | | | |
| AT1G30600 | Subtilase | 0.093 | 0.010 | 1.711 | 0.013 | 0.000 | 9.920 | 0.001 | 0.066 | 3.299 | | |
| | family protein | | | | | | | | | | | |
| AT5G67360 | Subtilase | 0.029 | 0.001 | 1.676 | 0.001 | 0.000 | 4.720 | 0.001 | 0.082 | 2.923 | | |
| | family protein | | | | | | | | | | | |
| AT1G76180 | Dehydrin | 0.062 | 0.005 | 1.659 | 0.017 | 0.010 | 3.048 | 0.001 | 0.080 | 2.975 | | |
| | family protein | | | | | | | | | | | |
| AT4G11740 | Ubiquitin-like | 0.084 | 0.008 | 1.653 | 0.000 | 0.000 | 7.747 | 0.001 | 0.067 | 3.272 | | |
| | superfamily | | | | | | | | | | | |
| | protein | | | | | | | | | | | |
| AT2G36910 | ATP binding | 0.012 | 0.000 | 1.569 | 0.000 | 0.001 | 3.596 | 0.001 | 0.092 | 2.693 | | |
| | cassette | | | | | | | | | | | |
| | subfamily B1 | | | | | | | | | | | |
| AT5G14930 | senescence- | 0.000 | 0.000 | 1.542 | 0.000 | 0.000 | 9.606 | 0.000 | 0.018 | 8.993 | | |
| | associated | | | | | | | | | | | |
| | gene 101 | | | | | | | | | | | |
| Significantly | | | | | | | | | | | | |
| induced by | | | | | | | | | | | | |
| HU and BM | | | | | | | | | | | | |
| AT5G66985 | unknown | 0.088 | 0.009 | 3.294 | | | | | | | 0.007 | 1.612 |
| | protein; Has | | | | | | | | | | | |
| | 30201 Blast | | | | | | | | | | | |
| | hits to 17322 | | | | | | | | | | | |
| | proteins in | | | | | | | | | | | |
| | 780 species: | | | | | | | | | | | |
| | Archae - 12; | | | | | | | | | | | |
| | Bacteria - 1396; | | | | | | | | | | | |
| | Metazoa - 17338; | | | | | | | | | | | |
| | Fungi - 3422; | | | | | | | | | | | |
| | Plants - 5037; | | | | | | | | | | | |
| | Viruses - 0; | | | | | | | | | | | |
| | Other | | | | | | | | | | | |
| | Eukaryotes - 2996 | | | | | | | | | | | |
| | (source: | | | | | | | | | | | |
| | NCBI BLink). | | | | | | | | | | | |
| AT5G14920 | Gibberellin- | 0.027 | 0.001 | 2.789 | | | | | | | 0.000 | 2.122 |
| | regulatcd | | | | | | | | | | | |
| | family protein | | | | | | | | | | | |
| AT4G15480 | UDP- | 0.081 | 0.008 | 2.196 | | | | | | | 0.000 | 2.394 |
| | Glycosyltransferase | | | | | | | | | | | |
| | superfamily | | | | | | | | | | | |
| | protein | | | | | | | | | | | |
| AT3G27620 | alternative | 0.077 | 0.007 | 2.056 | | | | | | | 0.025 | 1.883 |
| | oxidase 1C | | | | | | | | | | | |
| AT3G27950 | GDSL-like | 0.045 | 0.003 | 1.641 | | | | | | | 0.000 | 4.012 |
| | Lipase/Acylhydrolase | | | | | | | | | | | |
| | superfamily | | | | | | | | | | | |
| | protein | | | | | | | | | | | |
| AT4G04750 | Major | 0.082 | 0.008 | 1.625 | | | | | | | 0.011 | 1.689 |
| | facilitator | | | | | | | | | | | |
| | superfamily | | | | | | | | | | | |
| | protein | | | | | | | | | | | |
| AT5G60100 | pseudo- | 0.037 | 0.002 | 1.619 | | | | | | | 0.018 | 1.801 |
| | response | | | | | | | | | | | |
| | regulator 3 | | | | | | | | | | | |
| AT5G25810 | Integrase-type | 0.000 | 0.000 | 1.558 | | | | | | | 0.040 | 1.573 |
| | DNA-binding | | | | | | | | | | | |
| | superfamily | | | | | | | | | | | |
| | protein | | | | | | | | | | | |
| AT1G49030 | PLAC8 family | 0.044 | 0.003 | 1.553 | | | | | | | 0.000 | 2.653 |
| | protein | | | | | | | | | | | |
| Significantly | | | | | | | | | | | | |
| induced by | | | | | | | | | | | | |
| BM and | | | | | | | | | | | | |
| gamma rays | | | | | | | | | | | | |
| AT4G05370 | BCS1 AAA- | | | | 0.014 | 0.000 | 8.214 | 0.000 | 0.050 | 3.949 | 0.007 | 1.807 |
| | type ATPase | | | | | | | | | | | |
| AT5G49110 | unknown | | | | 0.004 | 0.001 | 7.611 | 0.000 | 0.037 | 4.819 | 0.002 | 1.562 |
| | protein; | | | | | | | | | | | |
| | INVOLVED | | | | | | | | | | | |
| | IN: biological | | | | | | | | | | | |
| | process | | | | | | | | | | | |
| | unknown; | | | | | | | | | | | |
| | LOCATED | | | | | | | | | | | |
| | IN: cellular | | | | | | | | | | | |
| | component | | | | | | | | | | | |
| | unknown; | | | | | | | | | | | |
| | EXPRESSED | | | | | | | | | | | |
| | IN: cultured | | | | | | | | | | | |
| | cell; Has | | | | | | | | | | | |
| | 30201 Blast | | | | | | | | | | | |
| | hits to 17322 | | | | | | | | | | | |
| | proteins in | | | | | | | | | | | |
| | 780 species: | | | | | | | | | | | |
| | Archae - 12; | | | | | | | | | | | |
| | Bacteria - 1396; | | | | | | | | | | | |
| | Metazoa - 17338; | | | | | | | | | | | |
| | Fungi - 3422; | | | | | | | | | | | |
| | Plants - 503 | | | | | | | | | | | |

^{a}According to Cools et al., 2011
^{b}According to Culligan et al., 2006
^{c}According to Yoshiyama et al., 2009

TABLE 3
Annotated Arabidopsis SIM/SMR genes

| AGI locus | Annotation |
|---|---|
| At5g04470 | SIM |
| At3g10525 | SMR1 |
| At1g08180 | SMR2 |
| At5g02420 | SMR3 |
| At5g02220 | SMR4 |
| At1g07500 | SMR5 |
| At5g40460 | SMR6 |
| At3g27630 | SMR7 |
| At1g10690 | SMR8 |
| At1g51355 | SMR9 |
| At2g28870 | SMR10 |
| At2g28330 | SMR11 |
| At2g37610 | SMR12 |
| At5g59360 | SMR13 |

TABLE 4
DNA ploidy level distribution in transgenic plants
overexpressing SMR4, SMR5, or SMR7

| Ploidy (%) | Col-0 | SMR4^{OE} | SMR5^{OE} | SMR7^{OE} |
|---|---|---|---|---|
| 2C | 19.6 ± 0.2 | 17.1 ± 0.1 | 23.6 ± 0.9 | 24.2 ± 1.3 |
| 4C | 26.3 ± 1.2 | 19.4 ± 0.5 | 21.3 ± 0.8 | 29.2 ± 0.7 |
| 8C | 49.2 ± 0.5 | 34.9 ± 3.4 | 34.8 ± 0.5 | 36.1 ± 0.2 |
| 16C | 4.6 ± 0.7 | 27.1 ± 3.1 | 19.6 ± 0.2 | 9.5 ± 0.9 |
| 32C | 0.2 ± 0 | 1.5 ± 0.6 | 0.7 ± 0.1 | 1.1 ± 0.1 |

TABLE 5
List of primers used for cloning, genotyping, and RT-PCR

Promoter cloning primers

| | | |
|---|---|---|
| SIAMESE | Fw | ATAGAAAAGTTGGTATTGTAATTATATATGAAAAAATAGTAAT |
| | | (SEQ ID NO: 7) |
| | Rev | GTACAAACTTGTTCTTTTTTGTTTATATAAATATTAAATGT |
| | | (SEQ ID NO: 8) |
| SMR1 | Fw | ATAGAAAAGTTGTCACAAGTGCATTTTTAATTTGTAGGA |
| | | (SEQ ID NO: 9) |
| | Rev | GTACAAACTTGCATCTAAACTTGTGTATGTTTTTGTTTTTTGG |
| | | (SEQ ID NO: 10) |
| SMR2 | Fw | ATAGAAAAGTTGGTAACTCCTTCGGCATCTTTGT (SEQ ID NO: 11) |
| | Rev | GTACAAACTTGTGGTCACATGGATGTGAAAGTTT (SEQ ID NO: 12) |
| SMR3 | Fw | ATAGAAAAGTTGGTATTTTAAATTACGATTTCAAAATCTTGA |
| | | (SEQ ID NO: 13) |
| | Rev | GTACAAACTTGTTAGACAAGTTTTACAGAGAGAAAGAAGAG |
| | | (SEQ ID NO: 14) |
| SMR4 | Fw | ATAGAAAAGTTGGTGAAACACAAAGCATCTTCG (SEQ ID NO: 15) |
| | Rev | GTACAAACTTGTTCTTCTCTCTCGAACTCG (SEQ ID NO: 16) |
| SMR5 | Fw | ATAGAAAAGTTGGTCAGAACGAACAAAAG (SEQ ID NO: 17) |
| | Rev | GTACAAACTTGTTTTTGTCCGCTCTCTCG (SEQ ID NO: 18) |
| SMR6 | Fw | ATAGAAAAGTTGGTCAGTGTGTCAAAACCGACG (SEQ ID NO: 19) |
| | Rev | GTACAAACTTGTCTCTCTTTAACTAACTCAAAACCAAGA |
| | | (SEQ ID NO: 20) |
| SMR7 | Fw | AGAAAAGTTGCGTTGACGCGGGAAAATTAA (SEQ ID NO: 21) |
| | Rev | GTACAAACTTGCTTAAAACAGTTGGAGATTGAG (SEQ ID NO: 22) |
| SMR8 | Fw | ATAGAAAAGTTGGTAGATCCCACATTACTTAAGAAATTGG |
| | | (SEQ ID NO: 23) |
| | Rev | GTACAAACTTGTGACTTCTCTCGAATGTGAATGAAGA (SEQ ID NO: 24) |
| SMR9 | Fw | ATAGAAAAGTTGGTACATATAAAGGTGTTATACACACCCTT |
| | | (SEQ ID NO: 25) |
| | Rev | GTACAAACTTGTTTTTGAGACCAGAATAAGAGAGAAG (SEQ ID NO: 26) |
| SMR10 | Fw | ATAGAAAAGTTGGTTTTAAAAAACCGTTTCAAACTAGTGC |
| | | (SEQ ID NO: 27) |
| | Rev | GTACAAACTTGTCTTTGAGAAGAAACGTCGCTC (SEQ ID NO: 28) |
| SMR11 | Fw | ATAGAAAAGTTGGTTGTGGTAATCTACATGGAATTTGC (SEQ ID NO: 29) |
| | Rev | GTACAAACTTGTTTGGATTCACGAGATCTAAGCA (SEQ ID NO: 30) |
| SMR12 | Fw | ATAGAAAAGTTGGTTCGGCTCACCTTGTTTTCC (SEQ ID NO: 31) |
| | Rev | GTACAAACTTGTGTGCGCTTTTTTTTCTTCTCAG (SEQ ID NO: 32) |
| SMR13 | Fw | ATAGAAAAGTTGGTAAAACTCAAGACACTTCTTTTTTTGG |
| | | (SEQ ID NO: 33) |
| | Rev | GTACAAACTTGTCTTATCACAAACAGGAAAAGAGAGAGT |
| | | (SEQ ID NO: 34) |

ORF cloning primers

| | | |
|---|---|---|
| SMR4 | Fw | AAAAAGCAGGCTTCATGGAGGTGG TGGAGAGGAA G (SEQ ID NO: 35) |
| | Rev + stop code | AGAAAGCTGGGTCCTAAGCGCAAGCTTCTCTTC (SEQ ID NO: 36) |
| | Rev - stop code | AGAAAGCTGGGTCAGCGCAAGCTTCTCTTC (SEQ ID NO: 37) |
| SMR5 | Fw | AAAAAGCAGGCTTCATGGAGGAGAAAAACTACGACG (SEQ ID NO: 38) |
| | Rev + stop code | AGAAAGCTGGGTCCTAGGTTGCCGCTTGGG (SEQ ID NO: 39) |
| | Rev - stop code | AGAAAGCTGGGTCGGTTGCCGCTTGGGA (SEQ ID NO: 40) |
| SMR7 | Fw | AAAAAGCAGGCTTCATGGGAATTTCGAAAAAATCTC (SEQ ID NO: 41) |
| | Rev + stop code | AGAAAGCTGGGTCTTAACGGCGTTGTATAAACACC (SEQ ID NO: 42) |
| | Rev - stop code | AGAAAGCTGGGTCACGGCGTTGTATAAACACCA (SEQ ID NO: 43) |

T-DNA genotyping primers

| | | | |
|---|---|---|---|
| SMR5 | SALK_100918 | LB | GAACGAACAAAAGTGAGCTCG (SEQ ID NO: 44) |
| | | RB | TTTCCCAACCTGACAGAAAAC (SEQ ID NO: 45) |
| SMR7 | SALK_128496 | LB | AAAATCGATAACTAAAACGAACCG (SEQ ID NO: 46) |
| | | RB | AGGCCTTCAATATAGCCCATG (SEQ ID NO: 47) |

RT-PCR primers

| | | |
|---|---|---|
| SIAMESE | Fw | CACAAGATTCCTCCCACCACAG (SEQ ID NO: 48) |
| | Rev | CAGAGGAGAAGAACCGCTCGAT (SEQ ID NO: 49) |
| SMR1 | Fw | CACCCACATCCCAAGAACACAAG (SEQ ID NO: 50) |
| | Rev | GACGGAGGAGAAGAAACGGTCAA (SEQ ID NO: 51) |
| SMR2 | Fw | AGAGCAGAAACCCAGAAGCCAAG (SEQ ID NO: 52) |
| | Rev | GAAATCTCACGCGGTCGCTTTCTT (SEQ ID NO: 53) |
| SMR3 | Fw | CGATCACAAGATTCCGGAGGTG (SEQ ID NO: 54) |
| | Rev | CGGCTCAGATCAATCGGTATGC (SEQ ID NO: 55) |
| SMR4 | Fw | GCCGAGAAGCACGATGTATAG (SEQ ID NO: 56) |
| | Rev | AGATCTGGTGGCTGAAAGTACC (SEQ ID NO: 57) |
| SMR5 | Fw | AAACTACGACGACGGAGATACG (SEQ ID NO: 58) |
| | Rev | GCTACCACCGAGAAGAACAAGT (SEQ ID NO: 59) |
| SMR6 | Fw | GGGCTTCGTTGAAACCAGTCAAG (SEQ ID NO: 60) |
| | Rev | TTTCTCGGTGCTGGTGGACATTC (SEQ ID NO: 61) |
| SMR7 | Fw | GCCAAAACATCGATTCGGGCTTC (SEQ ID NO: 62) |
| | Rev | TCGCCGTGGGAGTGATACAAAT (SEQ ID NO: 63) |
| SMR8 | Fw | TAACCTATCTCCCGGCGTCACA (SEQ ID NO: 64) |
| | Rev | GCACTTCAACGACGGTTTACGC (SEQ ID NO: 65) |
| SMR9 | Fw | GCCACTTCAAGAACCCATCTCC (SEQ ID NO: 66) |
| | Rev | TCCGGAGTACAACATCCACTCTCT (SEQ ID NO: 67) |
| SMR10 | Fw | GCAAAGAAGGAGCAACCGTCAAG (SEQ ID NO: 68) |
| | Rev | CGGTGGACAAATTCTTGGCATCG (SEQ ID NO: 69) |
| SMR11 | Fw | CTGCTTCGATCTCGGATTGTGTT (SEQ ID NO: 70) |
| | Rev | GACGAAGGAGGCGGTGTTTTAC (SEQ ID NO: 71) |
| SMR12 | Fw | GGTATGTCGGAGACGAGCTTGA (SEQ ID NO: 72) |
| | Rev | GAGTCGGTGTCTTGAACCCATCA (SEQ ID NO: 73) |
| SMR13 | Fw | GAACCACCAACACCGACAACAAG (SEQ ID NO: 74) |
| | Rev | GTTCGAGTTTCTCGGCGTCTCT (SEQ ID NO: 75) |
| Actin2 | Fw | GGCTCCTCTTAACCCAAAGGC (SEQ ID NO: 76) |
| | Rev | CACACCATCACCAGAATCCAGC (SEQ ID NO: 77) |
| EMB2386 | Fw | CTCTCGTTCCAGAGCTCGCAAAA (SEQ ID NO: 78) |
| | Rev | AAGAACACGCATCCTACGCATCC (SEQ ID NO: 79) |
| PAC1 | Fw | TCTCTTTGCAGGATGGGACAAGC (SEQ ID NO: 80) |
| | Rev | AGACTGAGCCGCCTGATTGTTTG (SEQ ID NO: 81) |
| RPS26C | Fw | GACTTTCAAGCGCAGGAATGGTG (SEQ ID NO: 82) |
| | Rev | CCTTGTCCTTGGGGCAACACTTT (SEQ ID NO: 83) |

### REFERENCES

Abraham, R. T. (2001). Cell cycle checkpoint signaling through the ATM and ATR kinases. Genes Dev. 15:2177-2196.
Adachi, S., K. Minamisawa, Y. Okushima, S. Inagaki, K. Yoshiyama, Y. Kondou, E. Kaminuma,
M. Kawashima, T. Toyoda, M. Matsui, D. Kurihara, S. Matsunaga, and M. Umeda (2011). Programmed induction of endoreduplication by DNA double-strand breaks in Arabidopsis. Proc. Natl. Acad. Sci. USA 108:10004-10009.
Amor, Y., E. Babiychuk, D. Inzé, and A. Levine (1998). The involvement of poly(ADP-ribose) polymerase in the oxidative stress responses in plants. FEBS Lett. 440:1-7.
Bartek, J., and J. Lukas (2001). Pathways governing G1/S transition and their response to DNA damage. FEBS Lett. 490:117-122.
Beeckman, T., and G. Engler (1994). An easy technique for the clearing of histochemically stained plant tissue. Plant Mol. Biol. Rep. 12:37-42.
Beers, R. F., Jr., and I. W. Sizer (1952). A spectrophotometric method for measuring the breakdown of hydrogen peroxide by catalase. J. Biol. Chem. 195:133-140.
Boudolf, V., K. Vlieghe, G. T. S. Beemster, Z. Magyar, J. A. Torres Acosta, S. Maes, E. Van Der Schueren, D. Inzé, and L. De Veylder (2004). The plant-specific cyclin-dependent kinase CDKB1;1 and transcription factor E2Fa-DPa control the balance of mitotically dividing and endoreduplicating cells in Arabidopsis. Plant Cell 16:2683-2692.
Boudolf, V., T. Lammens, J. Boruc, J. Van Leene, H. Van Den Daele, S. Maes, G. Van Isterdael, E. Russinova, E. Kondorosi, E. Witters, G. De Jaeger, D. Inzé, and L. De Veylder (2009). CDKB1;1 forms a functional complex with CYCA2;3 to suppress endocycle onset. Plant Physiol. 150:1482-1493.
Cadet, J., T. Douki, J.-L. Ravanat, and J. R. Wagner (2012). Measurement of oxidatively generated base damage to nucleic acids in cells: facts and artifacts. Bioanal. Rev. 4:55-74.
Chaturvedi, P., W. K. Eng, Y. Zhu, M. R. Mattern, R. Mishra, M. R. Hurle, X. Zhang, R. S. Annan, Q. Lu, L. F. Faucette, G. F. Scott, X. Li, S. A. Canrr, R. K. Johnson, J. D. Winkler, and B. B. Zhou (1999). Mammalian Chk2 is a downstream effector of the ATM-dependent DNA damage checkpoint pathway. Oncogene 18:4047-4054.
Che, F.-S., Y. Nakajima, N. Tanaka, M. Iwano, T. Yoshida, S. Takayama, I. Kadota, and A. Isogai (2000). Flagellin from an incompatible strain of Pseudomonas avenae induces a resistance response in cultured rice cells. J. Biol. Chem. 275:32347-32356.
Chen, G. X., and K. Asada (1990). Hydroxyurea and p-aminophenol are the suicide inhibitors of ascorbate peroxidase. J. Biol. Chem. 265:2775-2781.
Chen, Y., and Y. Sanchez (2004). Chk1 in the DNA damage response: conserved roles from yeasts to mammals. DNA Repair 3:1025-1032.
Churchman, M. L., M. L. Brown, N. Kato, V. Kirik, M. Hillskamp, D. Inzé, L. De Veylder, J. D. Walker, Z. Zheng, D. G. Oppenheimer, T. Gwin, J. Churchman, and J. C. Larkin (2006). SIAMESE, a plant-specific cell cycle regulator, controls endoreplication onset in Arabidopsis thaliana. Plant Cell 18:3145-3157.
Clough, S. J., and A. F. Bent (1998). Floral dip: a simplified method for Agrobacterium-mediated transformation of Arabidopsis thaliana. Plant J. 16:735-743.
Cools, T., A. Iantcheva, S. Maes, H. Van den Daele, and L. De Veylder (2010). A replication stress-induced synchronization method for Arabidopsis thaliana root meristems. Plant J. 64:705-714.
Cools, T., A. Iantcheva, A. K. Weimer, S. Boens, N. Takahashi, S. Maes, H. Van den Daele, G. Van Isterdael, A. Schnittger, and L. De Veylder (2011). The Arabidopsis thaliana checkpoint kinase WEE1 protects against premature vascular differentiation during replication stress. Plant Cell 23:1435-1448.
Culligan, K., A. Tissier, and A. Britt (2004). ATR regulates a G2-phase cell-cycle checkpoint in Arabidopsis thaliana. Plant Cell 16:1091-1104.
Culligan, K. M., C. E. Robertson, J. Foreman, P. Doerner, and A. B. Britt (2006). ATR and ATM play both distinct and additive roles in response to ionizing radiation. Plant J. 48:947-961.
De Clercq, A., and D. Inzé (2006). Cyclin-dependent kinase inhibitors in yeast, animals, and plants: a functional comparison. Crit. Rev. Biochem. Mol. Biol. 41:293-313.
De Schutter, K., J. Joubes, T. Cools, A. Verkest, F. Corellou, E. Babiychuk, E. Van Der Schueren, T. Beeckman, S. Kushnir, D. Inzé, and L. De Veylder (2007). Arabidopsis WEE1 kinase controls cell cycle arrest in response to activation of the DNA integrity checkpoint. Plant Cell 19:211-225.
De Veylder, L., G. Segers, N. Glab, P. Casteels, M. Van Montagu, and D. Inzé (1997). The Arabidopsis Cks1At protein binds the cyclin-dependent kinases Cdc2aAt and Cdc2bAt. FEBS Lett. 412:446-452.
De Veylder, L., T. Beeckman, G. T. S. Beemster, L. Krols, F. Terms, I. Landrieu, E. Van Der Schueren, S. Maes, M. Naudts, and D. Inzé (2001). Functional analysis of cyclin-dependent kinase inhibitors of Arabidopsis. Plant Cell 13:1653-1667.
DePaoli, H. C., G. H. Goldman, and M.-H. S. Goldman (2012). SCI1, the first member of the tissue-specific inhibitors of CDK (TIC) class, is probably connected to the auxin signaling pathway. Plant Signal. Behav. 7:53-58.
DePaoli, H. C., M. S. Brito, A. C. Quiapim, S. P. Teixeira, G. H. Goldman, M. C. Dornelas, and M. H. S. Goldman (2011). Stigma/style cell cycle inhibitor 1 (SCI1), a tissue-specific cell cycle regulator that controls upper pistil development. New Phytol. 190:882-895.
Dewitte, W., and J. A. H. Murray (2003). The plant cell cycle. Annu. Rev. Plant Biol. 54:235-264.
Dissmeyer, N., A. K. Weimer, S. Pusch, K. De Schutter, C. L. Alvim Kamei, M. K. Nowack, B. Novak, G.-L. Duan, Y.-G. Zhu, L. De Veylder, and A. Schnittger (2009). Control of cell proliferation, organ growth, and DNA damage response operate independently of dephosphorylation of the Arabidopsis Cdk1 homolog CDKA;1. Plant Cell 21:3641-3654.
Dizdaroglu, M., P. Jaruga, M. Birincioglu, and H. Rodriguez (2002). Free radical-induced damage to DNA: mechanisms and measurement. Free Radic. Biol. Med. 32:1102-1115.
Dubacq, C., A. Chevalier, R. Courbeyrette, C. Petat, X. Gidrol, and C. Mann (2006). Role of the iron mobilization and oxidative stress regulons in the genomic response of yeast to hydroxyurea. Mol. Genet. Genomics 275:114-124.
Dunand, C., M. Crevecoeur, and C. Penel (2007). Distribution of superoxide and hydrogen peroxide in Arabidopsis root and their influence on root development: possible interaction with peroxidases. New Phytol. 174:332-341.
Francis, D. (2011). A commentary on the G2/M transition of the plant cell cycle. Ann. Bot. 107:1065-1070.
French, A. P., M. H. Wilson, K. Kenobi, D. Dietrich, U. VoB, S. Ubeda-Tomas, T. P. Pridmore, and D. M. Wells (2012). Identifying biological landmarks using a novel cell measuring image analysis tool: Cell-o-Tape. Plant Methods 8:7.
Gaj, T., C. A. Gersbach, and C. F. BARBAS III (2013). ZFN, TALEN and CRISPR/Cas-based methods for genome engineering. Trends Biotechnol. 31:397-405.
Galbraith, D. W., K. R. Harkins, and S. Knapp (1991). Systemic Endopolyploidy in Arabidopsis thaliana. Plant Physiol. 96:985-989.
Garcia, V., H. Bruchet, D. Camescasse, F. Granier, D. Bouchez, and A. Tissier (2003). AtATM is essential for meiosis and the somatic response to DNA damage in plants. Plant Cell 15:119-132.
Gendrel, A.-V., Z. Lippman, R. Martienssen, and V. Colot (2005). Profiling histone modification patterns in plants using genomic tiling microarrays. Nat. Methods 2:213-218.
Guo, Z., S. Kozlov, M. F. Lavin, M. D. Person, and T. T. Paull (2010). ATM activation by oxidative stress. Science 330:517-521.
Hruz, T., O. Laule, G. Szabo, F. Wessendorp, S. Bleuler, L. Oertle, P. Widmayer, W. Gruissem, and P. Zimmermann (2008). GENEVESTIGATOR® V3: a reference expression database for the meta-analysis of transcriptomes. Adv. Bioinformatics 2008:420747.
Irizarry, R. A., B. Hobbs, F. Collin, Y. D. Beazer-Barclay, K. J. Antonellis, U. Scherf, and T. P. Speed (2003). Exploration, normalization, and summaries of high density oligonucleotide array probe level data. Biostatistics 4:249-264.
Juul, T., A. Malolepszy, K. Dybkær, R. Kidmose, J. T. Rasmussen, G. R. Andersen, H. E. Johnsen, J.-E. Jorgensen, and S. U. Andersen (2010). The in vivo toxicity of hydroxyurea depends on its direct target catalase. J. Biol. Chem. 285:21411-21415.
Karimi, M., A. Bleys, R. Vanderhaeghen, and P. Hilson (2007). Building blocks for plant gene assembly. Plant Physiol. 145:1183-1191.
Karimi, M., D. Inzé, and A. Depicker (2002). GATEWAY™ vectors for Agrobacterium-mediated plant transformation. Trends Plant Sci. 7:193-195.
Kinoshita, E., E. Kinoshita-Kikuta, K. Takiyama, and T. Koike (2006). Phosphate-binding tag, a new tool to visualize phosphorylated proteins. Mol. Cell. Proteomics 5:749-757.
Kurz, E. U., and S. P. Lees-Miller (2004). DNA damage-induced activation of ATM and ATM-dependent signaling pathways. DNA Repair 3:889-900.
Lee, M. G., and P. Nurse (1987). Complementation used to clone a human homologue of the fission yeast cell cycle control gene cdc2. Nature 327:31-35.
Minami, E., K. Kuchitsu, D.-Y. He, H. Kouchi, N. Midoh, Y. Ohtsuki, and H. Shibuya (1996). Two novel genes rapidly and transiently activated in suspension-cultured rice cells by treatment with N-acetylchitoheptaose, a biotic elicitor for phytoalexin production. Plant Cell Physiol. 37:563-567.
Mittler, R., S. Vanderauwera, M. Gollery, and F. Van Breusegem (2004). Reactive oxygen gene network of plants. Trends Plant Sci. 9:490-498.
Mittler, R., S. Vanderauwera, N. Suzuki, G. Miller, V. B. Tognetti, K. Vandepoele, M. Gollery, V. Shulaev, and F. Van Breusegem (2011). ROS signaling: the new wave? Trends Plant Sci. 16:300-309.
Murashige, T., and F. Skoog (1962). A revised medium for rapid growth and bio assays with tobacco tissue cultures. Physiol. Plant. 15:473-497.
Murata, M., K. Fukushima, T. Takao, H. Seki, S. Takeda, and N. Wake (2013). Oxidative stress produced by xanthine oxidase induces apoptosis in human extravillous trophoblast cells. J. Reprod. Dev. 59:7-13.
Norbury, C., and P. Nurse (1992). Animal cell cycles and their control. Annu. Rev. Biochem. 61:441-468.
Nowack, M. K., A. Ungru, K. N. Bjerkan, P. E. Grini, and A. Schnittger (2010). Reproductive cross-talk: seed development in flowering plants. Biochem. Soc. Trans. 38:604-612.
Nowack, M. K., H. Harashima, N. Dissmeyer, X. Zhao, D. Bouyer, A. K. Weimer, F. De Winter, F. Yang, and A. Schnittger (2012). Genetic framework of cyclin-dependent kinase function in Arabidopsis. Dev. Cell 22:1030-1040.
Nyholm, S., G. J. Mann, A. G. Johansson, R. J. Bergeron, A. Grislund, and L. Thelander (1993). Role of ribonucleotide reductase in inhibition of mammalian cell growth by potent iron chelators. J. Biol. Chem. 268:26200-26205.
Peres, A., M. L. Churchman, S. Hariharan, K. Himanen, A. Verkest, K. Vandepoele, Z. Magyar, Y. Hatzfeld, E. Van Der Schueren, G. T. S. Beemster, V. Frankard, J. C. Larkin, D. Inzé, and L. De Veylder (2007). Novel plant-specific cyclin-dependent kinase inhibitors induced by biotic and abiotic stresses. J. Biol. Chem. 282:25588-25596.
Preuss, S. B., and A. B. Britt (2003). A DNA-damage-induced cell cycle checkpoint in Arabidopsis. Genetics 164:323-334.
Reichheld, J.-P., T. Vernoux, F. Lardon, M. Van Montagu, and D. Inzé (1999). Specific checkpoints regulate plant cell cycle progression in response to oxidative stress. Plant J. 17:647-656.
Ricaud, L., C. Proux, J.-P. Renou, O. Pichon, S. Fochesato, P. Ortet, and M.-H. Montané (2007). ATM-mediated transcriptional and developmental responses to 7-rays in Arabidopsis. PLoS ONE 2:e430.
Riou-Khamlichi, C., M. Menges, J. M. S. Healy, and J. A. H. Murray (2000). Sugar control of the plant cell cycle: differential regulation of Arabidopsis D-type cyclin gene expression. Mol. Cell. Biol. 20:4513-4521.
Roeder, A. H. K., V. Chickarmane, A. Cunha, B. Obara, B. S., Manjunath, and E. M. Meyerowitz (2010). Variability in the control of cell division underlies sepal epidermal patterning in Arabidopsis thaliana. PLoS Biol. 8:e1000367.
Roldan-Arjona, T., and R. R. Ariza (2009). Repair and tolerance of oxidative DNA damage in plants. Mutat. Res. 681:169-179.
Rozan, L. M., and W. S. El-Deiry (2007). p53 downstream target genes and tumor suppression: a classical view in evolution. Cell Death Differ. 14:3-9.
Sherr, C. J., and J. M. Roberts (1995). Inhibitors of mammalian G1 cyclin-dependent kinases. Genes Dev. 9:1149-1163.
Shieh, S.-Y., J. Ahn, K. Tamai, Y. Taya, and C. Prives (2000). The human homologs of checkpoint kinases Chk1 and Cds1 (Chk2) phosphorylate p53 at multiple DNA damage-inducible sites. Genes Dev. 14:289-300.
Tsukagoshi, H. (2012). Defective root growth triggered by oxidative stress is controlled through the expression of cell cycle-related genes. Plant Sci. 197:30-39.
Tsukagoshi, H., W. Busch, and P. N. Benfey (2010). Transcriptional regulation of ROS controls transition from proliferation to differentiation in the root. Cell 143:606-616.
Tusher, V. G., R. Tibshirani, and G. Chu (2001). Significance analysis of microarrays applied to the ionizing radiation response. Proc. Natl. Acad. Sci. USA 98:5116-5121.
Van Leene, J., J. Hollunder, D. Eeckhout, G. Persiau, E. Van De Slijke, H. Stals, G. Van Isterdael, A. Verkest, S. Neirynck, Y. Buffel, S. De Bodt, S. Maere, K. Laukens, A. Pharazyn, P. C. G. Ferreira, N. Eloy, C. Renne, C. Meyer, J.-D. Faure, J. Steinbrenner, J. Beynon, J. C. Larkin, Y. Van de Peer, P. Hilson, M. Kuiper, L. De Veylder, H. Van Onckelen, D. Inzé, E. Witters, and G. De Jaeger (2010). Targeted interactomics reveals a complex core cell cycle machinery in Arabidopsis thaliana. Mol. Syst. Biol. 6:397.
Vandenabeele, S., S. Vanderauwera, M. Vuylsteke, S. Rombauts, C. Langebartels, H. K. Seidlitz, M. Zabeau, M. Van Montagu, D. Inzé, and F. Van Breusegem (2004). Catalase deficiency drastically affects gene expression induced by high light in Arabidopsis thaliana. Plant J. 39:45-58.
Vanderauwera, S., N. Suzuki, G. Miller, B. van de Cotte, S. Morsa, J.-L. Ravanat, A. Hegie, C. Triantaphylides, V. Shulaev, M. C. E. Van Montagu, F. Van Breusegem, and R. Mittler (2011). Extranuclear protection of chromosomal DNA from oxidative stress. Proc. Natl. Acad. Sci. USA 108:1711-1716.
Vernoux, T., R. C. Wilson, K. A. Seeley, J.-P. Reichheld, S. Muroy, S. Brown, S. C. Maughan, C. S. Cobbett, M. Van Montagu, D. Inzé, M. J. May, and Z. R. Sung (2000). The ROOT MERISTEMLESS1/CADMIUM SENSITIVE2 gene defines a glutathione-dependent pathway involved in initiation and maintenance of cell division during postembryonic root development. Plant Cell 12:97-109.
Wang, H., Y. Zhou, S. Gilmer, S. Whitwill, and L. C. Fowke (2000). Expression of the plant cyclin-dependent kinase inhibitor ICK1 affects cell division, plant growth and morphology. Plant J. 24:613-623.
Wang, H., Q. Qi, P. Schorr, A. J. Cutler, W. L. Crosby, and L. C. Fowke (1998). ICK1, a cyclin-dependent protein kinase inhibitor from Arabidopsis thaliana interacts with both Cdc2a and CycD3, and its expression is induced by abscisic acid. Plant J. 15:501-510.
Yoshiyama, K., P. A. Conklin, N. D. Huefner, and A. B. Britt (2009). Suppressor of gamma response 1 (SOG1) encodes a putative transcription factor governing multiple responses to DNA damage. Proc. Natl. Acad. Sci. USA 106:12843-12848.
Yoshiyama, K. O., J. Kobayashi, N. Ogita, M. Ueda, S. Kimura, H. Maki, and M. Umeda (2013). ATM-mediated phosphorylation of SOG1 is essential for the DNA damage response in Arabidopsis. EMBO Rep. 14:817-822.
Zhou, B.-B., and S. J. Elledge (2000). The DNA damage response: putting checkpoints in perspective. Nature 408:433-439.

1. A method of modulating reactive oxygen species (ROS) signaling and/or oxidative stress in a plant, the method comprising:
utilizing SMR5 to modulate ROS signaling and/or oxidative stress response in the plant.

2. The method according to claim 1, wherein SMR5 encodes a protein selected from the group consisting of SEQ ID NO:2, SEQ ID NO:4 and SEQ ID NO:6.

3. The method according to claim 1, wherein utilizing SMR5 is a down-regulation of SMR5 expression.

4. The method according to claim 1, wherein oxidative stress tolerance is increased in the plant.

5. The method according to claim 1, further comprising:
down-regulating SMR4 and/or SMR7 so as to increase oxidative stress tolerance in the plant.

6. A genetically modified plant, comprising an inactivated SMR5 gene and/or protein.

7. The genetically modified plant according to claim 6, wherein the plant further comprises:
an inactivated SMR4 gene and/or protein, and/or
an inactivated SMR7 gene and/or protein.

8. A method of increasing oxidative stress resistance in a plant, the method comprising:
down-regulating SMR5p expression and/or activity.

9. The method according to claim 8, further comprising down-regulating SMR4p and/or SMR7p expression and/or activity in the plant.

10. The method according to claim 2, wherein utilizing SMR5 comprises down-regulating SMR5 expression in the plant.

11. The method according to claim 2, wherein oxidative stress tolerance is increased in the plant.

12. The method according to claim 3, wherein oxidative stress tolerance is increased in the plant.

13. The method according to claim 1, further comprising:
down-regulating SMR4 gene in the plant so as to increase oxidative stress tolerance in the plant.

14. The method according to claim 1, further comprising:
down-regulating SMR7 gene in the plant so as to improve oxidative stress tolerance in the plant.

15. A genetically modified plant having an increased resistance to oxidative stress in comparison to a wild-type of the genetically modified plant, the genetically modified plant comprising:
an inactivated or down-regulated SMR5 gene.

16. The genetically modified plant of claim 15, further comprising:
an inactivated or down-regulated SMR4 gene.

17. The genetically modified plant of claim 15, further comprising:
an inactivated or down-regulated SMR7 gene.

18. The genetically modified plant of claim 15, further comprising:
an inactivated or down-regulated SMR4 gene, and
an inactivated or down-regulated SMR7 gene.

19. The genetically modified plant of claim 15, wherein the SMR5 gene encodes a protein selected from the group consisting of SEQ ID NO:2, SEQ ID NO:4, and SEQ ID NO:6.

20. The genetically modified plant of claim 18, wherein the SMR5 gene encodes a protein selected from the group consisting of SEQ ID NO:2, SEQ ID NO:4, and SEQ ID NO:6.


SEQUENCE LISTING (83 sequences)

SEQ ID NO: 1 - DNA, 881 bp, Arabidopsis thaliana
  CDS (91)..(336)
attttgactgatagtgacctgttcgttgcacaaattgatgagcaatgcttttttataatgccatttttgtacaaaaaagcaggcttcatggaggagaaaaactacgacgacggagatacggtgacggttgatgatgattatcagatgggatgcacgacgcctacacgtgatgattgccggataccagcatatccgccttgtccacctccggtgagaaggaagagatcgctactaggctttgggaagaagagggaaccaccgaagaagggatattttcagccgccggatctagacttgttcttctcggtggtagcagcctcccaagcggcaacctaggacccagctttcttgtacaaagttggcattataagaaagcattgcttatcaatttgttgcaacgaacaggtcactatcagtcaaaataaaatcattatttgccatccggctgatatcccctatagtgagtcgtattacatggtcatagctgtttcctggcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgc

SEQ ID NO: 2 - protein, 81 aa, Arabidopsis thaliana
EEKNYDDGDTVTVDDDYQMGCTTPTRDDCRIPAYPPCPPPVRRKRSLLGFGKKREPPKKGYFQPPDLDLFFSVVAASQAAT

SEQ ID NO: 3 - DNA, 375 bp, Zea mays
  CDS (1)..(375)
atgccccgccagccgcgtctcttccctcgcaaacccacgtttcaaaaaaaagagaggaaagcaaagttccttcttccctcgaaaaaaaaaatcagtctcgccatggagagcagcgttggcatagagaaggccgcagcggtggcggttggtgcaggtgtgggcgggggaggtggagggtacggctgcggcgggtgggagacgccgaagcgcgaggagtgccgcatcccggcgacgctgccgtgccccgcggcgccgaggaaggccgtgccggacttcgggaagcggcgcagcccgcccaagaacggctacttccagccgccggacctggaggcgctcttcgcgctcgcgccgcgccgccaggccttctgcgcgtga

SEQ ID NO: 4 - protein, 124 aa, Zea mays
MPRQPRLFPRKPTFQKKERKAKFLLPSKKKISLAMESSVGIEKAAAVAVGAGVGGGGGGYGCGGWETPKREECRIPATLPCPAAPRKAVPDFGKRRSPPKNGYFQPPDLEALFALAPRRQAFCA

SEQ ID NO: 5 - DNA, 288 bp, Zea mays
  CDS (1)..(288)
atggaggtggagcacggcggagagatgatgatgttggtcgaggcggctgcggcggatgaggaggggtggcagacgccgaggcgcgaggactgccgcatccctgtggtgccgccgtgcccggcggcgccgtcgaggaagaaggccgtcgcgatggcgccggaggcggccggagggagcaggcggcgggacccgcccaagggcgggtacttccagccaccggacctcgagtccctgttcgtgctcgcgccgccgagggcacaggcggcggcctcaagccgcgcgtggtaa

SEQ ID NO: 6 - protein, 95 aa, Zea mays
MEVEHGGEMMMLVEAAAADEEGWQTPRREDCRIPVVPPCPAAPSRKKAVAMAPEAAGGSRRRDPPKGGYFQPPDLESLFVLAPPRAQAAASSRAW

SEQ ID NO: 7 - DNA, 43 bp, artificial
  Primer
atagaaaagttggtattgtaattatatatgaaaaaatagtaat

SEQ ID NO: 8 - DNA, 41 bp, artificial
  Primer
gtacaaacttgttcttttttgtttatataaatattaaatgt

SEQ ID NO: 9 - DNA, 39 bp, artificial
  Primer
atagaaaagttgtcacaagtgcatttttaatttgtagga

SEQ ID NO: 10 - DNA, 43 bp, artificial
  Primer
gtacaaacttgcatctaaacttgtgtatgtttttgttttttgg

SEQ ID NO: 11 - DNA, 34 bp, artificial
  Primer
atagaaaagttggtaactccttcggcatctttgt

SEQ ID NO: 12 - DNA, 34 bp, artificial
  Primer
gtacaaacttgtggtcacatggatgtgaaagttt

SEQ ID NO: 13 - DNA, 42 bp, artificial
  Primer
atagaaaagttggtattttaaattacgatttcaaaatcttga

SEQ ID NO: 14 - DNA, 41 bp, artificial
  Primer
gtacaaacttgttagacaagttttacagagagaaagaagag

SEQ ID NO: 15 - DNA, 33 bp, artificial
  Primer
atagaaaagttggtgaaacacaaagcatcttcg

SEQ ID NO: 16 - DNA, 30 bp, artificial
  Primer
gtacaaacttgttcttctctctcgaactcg

SEQ ID NO: 17 - DNA, 29 bp, artificial
  Primer
atagaaaagttggtcagaacgaacaaaag

SEQ ID NO: 18 - DNA, 29 bp, artificial
  Primer
gtacaaacttgtttttgtccgctctctcg

SEQ ID NO: 19 - DNA, 33 bp, artificial
  Primer
atagaaaagttggtcagtgtgtcaaaaccgacg

SEQ ID NO: 20 - DNA, 39 bp, artificial
  Primer
gtacaaacttgtctctctttaactaactcaaaaccaaga

SEQ ID NO: 21 - DNA, 30 bp, artificial
  Primer
agaaaagttgcgttgacgcgggaaaattaa

SEQ ID NO: 22 - DNA, 33 bp, artificial
  Primer
gtacaaacttgcttaaaacagttggagattgag

SEQ ID NO: 23 - DNA, 40 bp, artificial
  Primer
atagaaaagttggtagatcccacattacttaagaaattgg

SEQ ID NO: 24 - DNA, 37 bp, artificial
  Primer
gtacaaacttgtgacttctctcgaatgtgaatgaaga

SEQ ID NO: 25 - DNA, 41 bp, artificial
  Primer
atagaaaagttggtacatataaaggtgttatacacaccctt

SEQ ID NO: 26 - DNA, 37 bp, artificial
  Primer
gtacaaacttgtttttgagaccagaataagagagaag

SEQ ID NO: 27 - DNA, 40 bp, artificial
  Primer
atagaaaagttggttttaaaaaaccgtttcaaactagtgc

SEQ ID NO: 28 - DNA, 33 bp, artificial
  Primer
gtacaaacttgtctttgagaagaaacgtcgctc

SEQ ID NO: 29 - DNA, 38 bp, artificial
  Primer
atagaaaagttggttgtggtaatctacatggaatttgc

SEQ ID NO: 30 - DNA, 34 bp, artificial
  Primer
gtacaaacttgtttggattcacgagatctaagca

SEQ ID NO: 31 - DNA, 33 bp, artificial
  Primer
atagaaaagttggttcggctcaccttgttttcc

SEQ ID NO: 32 - DNA, 34 bp, artificial
  Primer
gtacaaacttgtgtgcgcttttttttcttctcag

SEQ ID NO: 33 - DNA, 40 bp, artificial
  Primer
atagaaaagttggtaaaactcaagacacttctttttttgg

SEQ ID NO: 34 - DNA, 39 bp, artificial
  Primer
gtacaaacttgtcttatcacaaacaggaaaagagagagt

SEQ ID NO: 35 - DNA, 35 bp, artificial
  Primer
aaaaagcaggcttcatggaggtggtggagaggaag

SEQ ID NO: 36 - DNA, 33 bp, artificial
  Primer
agaaagctgggtcctaagcgcaagcttctcttc

SEQ ID NO: 37 - DNA, 30 bp, artificial
  Primer
agaaagctgggtcagcgcaagcttctcttc

SEQ ID NO: 38 - DNA, 36 bp, artificial
  Primer
aaaaagcaggcttcatggaggagaaaaactacgacg

SEQ ID NO: 39 - DNA, 30 bp, artificial
  Primer
agaaagctgggtcctaggttgccgcttggg

SEQ ID NO: 40 - DNA, 28 bp, artificial
  Primer
agaaagctgggtcggttgccgcttggga

SEQ ID NO: 41 - DNA, 36 bp, artificial
  Primer
aaaaagcaggcttcatgggaatttcgaaaaaatctc

SEQ ID NO: 42 - DNA, 35 bp, artificial
  Primer
agaaagctgggtcttaacggcgttgtataaacacc

SEQ ID NO: 43 - DNA, 33 bp, artificial
  Primer
agaaagctgggtcacggcgttgtataaacacca

SEQ ID NO: 44 - DNA, 21 bp, artificial
  Primer
gaacgaacaaaagtgagctcg

SEQ ID NO: 45 - DNA, 21 bp, artificial
  Primer
tttcccaacctgacagaaaac

SEQ ID NO: 46 - DNA, 24 bp, artificial
  Primer
aaaatcgataactaaaacgaaccg

SEQ ID NO: 47 - DNA, 21 bp, artificial
  Primer
aggccttcaatatagcccatg

SEQ ID NO: 48 - DNA, 22 bp, artificial
  Primer
cacaagattcctcccaccacag

SEQ ID NO: 49 - DNA, 22 bp, artificial
  Primer
cagaggagaagaaccgctcgat

SEQ ID NO: 50 - DNA, 23 bp, artificial
  Primer
cacccacatcccaagaacacaag

SEQ ID NO: 51 - DNA, 23 bp, artificial
  Primer
gacggaggagaagaaacggtcaa

SEQ ID NO: 52 - DNA, 23 bp, artificial
  Primer
agagcagaaacccagaagccaag

SEQ ID NO: 53 - DNA, 24 bp, artificial
  Primer
gaaatctcacgcggtcgctttctt

SEQ ID NO: 54 - DNA, 22 bp, artificial
  Primer
cgatcacaagattccggaggtg

SEQ ID NO: 55 - DNA, 22 bp, artificial
  Primer
cggctcagatcaatcggtatgc

SEQ ID NO: 56 - DNA, 21 bp, artificial
  Primer
gccgagaagcacgatgtatag

SEQ ID NO: 57 - DNA, 22 bp, artificial
  Primer
agatctggtggctgaaagtacc

SEQ ID NO: 58 - DNA, 22 bp, artificial
  Primer
aaactacgacgacggagatacg

SEQ ID NO: 59 - DNA, 22 bp, artificial
  Primer
gctaccaccgagaagaacaagt

SEQ ID NO: 60 - DNA, 23 bp, artificial
  Primer
gggcttcgttgaaaccagtcaag

SEQ ID NO: 61 - DNA, 23 bp, artificial
  Primer
tttctcggtgctggtggacattc

SEQ ID NO: 62 - DNA, 23 bp, artificial
  Primer
gccaaaacatcgattcgggcttc

SEQ ID NO: 63 - DNA, 22 bp, artificial
  Primer
tcgccgtgggagtgatacaaat

SEQ ID NO: 64 - DNA, 22 bp, artificial
  Primer
taacctatctcccggcgtcaca

SEQ ID NO: 65 - DNA, 22 bp, artificial
  Primer
gcacttcaacgacggtttacgc

SEQ ID NO: 66 - DNA, 22 bp, artificial
  Primer
gccacttcaagaacccatctcc

SEQ ID NO: 67 - DNA, 24 bp, artificial
  Primer
tccggagtacaacatccactctct

SEQ ID NO: 68 - DNA, 23 bp, artificial
  Primer
gcaaagaaggagcaaccgtcaag

SEQ ID NO: 69 - DNA, 23 bp, artificial
  Primer
cggtggacaaattcttggcatcg

SEQ ID NO: 70 - DNA, 23 bp, artificial
  Primer
ctgcttcgatctcggattgtgtt

SEQ ID NO: 71 - DNA, 22 bp, artificial
  Primer
gacgaaggaggcggtgttttac

SEQ ID NO: 72 - DNA, 22 bp, artificial
  Primer
ggtatgtcggagacgagcttga

SEQ ID NO: 73 - DNA, 23 bp, artificial
  Primer
gagtcggtgtcttgaacccatca

SEQ ID NO: 74 - DNA, 23 bp, artificial
  Primer
gaaccaccaacaccgacaacaag

SEQ ID NO: 75 - DNA, 22 bp, artificial
  Primer
gttcgagtttctcggcgtctct

SEQ ID NO: 76 - DNA, 21 bp, artificial
  Primer
ggctcctcttaacccaaaggc

SEQ ID NO: 77 - DNA, 22 bp, artificial
  Primer
cacaccatcaccagaatccagc

SEQ ID NO: 78 - DNA, 23 bp, artificial
  Primer
ctctcgttccagagctcgcaaaa

SEQ ID NO: 79 - DNA, 23 bp, artificial
  Primer
aagaacacgcatcctacgcatcc

SEQ ID NO: 80 - DNA, 23 bp, artificial
  Primer
tctctttgcaggatgggacaagc

SEQ ID NO: 81 - DNA, 23 bp, artificial
  Primer
agactgagccgcctgattgtttg

SEQ ID NO: 82 - DNA, 23 bp, artificial
  Primer
gactttcaagcgcaggaatggtg

SEQ ID NO: 83 - DNA, 23 bp, artificial
  Primer
ccttgtccttggggcaacacttt